(19) United States
(12) Patent Application Publication
(10) Pub. No.: US 2006/0064119 A9
(43) Pub. Date: Mar. 23, 2006

(54) Vascular anastomosis systems

(75) Inventors: Alexander Q. Tilson, Burlingame, CA (US); James G. Whayne, Chapel Hill, NC (US); Patrick M. Owens, Half Moon Bay, CA (US); Jeffery Byron Alvarez, Brisbane, CA (US); Mitchell Craig Barham, Cupertino, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Converge Medical, Inc.

(21) Appl. No.: 10/963,978

(22) Filed: Oct. 12, 2004

Related U.S. Application Data
(63) Continuation-in-part of application No. 10/810,315, filed on Mar. 25, 2004, now abandoned.
Application No. 10/810,315 is a continuation-in-part of application No. 10/187,655, filed on Jul. 1, 2002.
Application No. 10/810,315 is a continuation-in-part of application No. 09/991,469, filed on Nov. 21, 2001, now Pat. No. 6,858,035.
Application No. 09/991,469 is a continuation-in-part of application No. 09/899,346, filed on Jul. 5, 2001, now Pat. No. 6,626,920.
(60) Provisional application No. 60/387,824, filed on Jun. 10, 2002.

(51) Int. Cl. A61B 17/08

(52) U.S. Cl. 606/153

(57) ABSTRACT

Devices and methods are described herein for implanting a connector to provide an anastomotic connection at a graft/host vessel junction.

### CROSS-REFERENCE TO RELATED APPLICATIONS

This application is a continuation-in-part of U.S. patent Ser. No. 10/810,315 filed Mar. 25, 2004 which is a continuation-in-part of U.S. patent Ser. No. 10/187,655, filed Jul. 1, 2002, which claims the benefit of priority to U.S. Provisional Patent Application Ser. No. 60/387,824, filed Jun. 10, 2002, and is also a continuation-in-part of U.S. patent application Ser. No. 09/991,469, filed Nov. 21, 2001, which is a continuation-in-part of U.S. patent application Ser. No. 09/899,346, filed Jul. 5, 2001, now U.S. Pat. No. 6,626,920, all the disclosures of which are incorporated herein by reference in their entirety.

### FIELD OF THE INVENTION

This relates to producing end-to-side anastomoses, particularly in communication with coronary arteries, the aorta, the subclavian, iliacs, femoral arteries, popliteal arteries, radial arteries, mammary arteries, mesenteric arteries, renal arteries, carotid arteries, cerebral arteries, or other tubular structures. Accordingly, angled anastomosis connectors and associated devices are disclosed.

### BACKGROUND OF THE INVENTION

This invention provides devices and methods to position and secure bypass grafts at host vessel locations without having to stop or re-route blood flow for extended periods of time, which is a condition of conventional sutured anastomoses. In addition, this invention reproducibly creates angled anastomoses between bypass grafts and host vessels thereby optimizing flow dynamics through the anastomoses and mitigating risks associated with suturing, clipping or stapling the bypass graft to a host vessel, namely reduction of anastomotic opening or excessive bleeding from the puncture holes. These risks may be mitigated, in part, by features adapted to avoid bleeding at graft attachment sites and preventing the host vessel from collapsing around the incision point.

In performing cardiac bypass surgery, anastomosis sites are typically provided at a site along a patient's aorta, and another site along a coronary artery beyond a partial or complete occlusion. Alternatively, sequential “jumper” grafts may extend from a main bypass graft to individual coronary artery host vessels thereby requiring a single aortic anastomosis to accommodate multiple coronary anastomoses. As such, in-flow anastomoses are required along the main “feeder” graft and out-flow anastomoses are required to the host vessel coronary arteries. This eliminates the need for side-side anastomoses between a single graft and multiple coronary arteries when producing sequential anastomoses from a single aortic anastomosis. Producing an effective anastomosis along a coronary artery is particularly challenging. The outer diameter of a coronary artery where a distal anastomosis may be needed can range from between about 1 mm to about 4 mm in size. By way of comparison, the outer diameter of the aorta where a proximal anastomosis may be located ranges between about 20 mm and about 50 mm in size.

The relatively small size of the site for a distal anastomosis translates to greater difficulty in a number of ways. Basic surgical challenges are encountered in dealing with the smaller vasculature. Further, an interface issue is introduced. Often, particularly for connection with the smaller coronary arteries, a graft conduit will have a larger diameter than the host vessel. This may be due to the need for a larger diameter conduit to carry adequate blood flow or the result of using a saphenous vein which must be oriented so its valving allows blood to readily flow in the desired direction from the proximal anastomosis to the distal anastomosis, thereby orienting the larger end of the graft toward the distal site. For whatever reason, the mis-match in size in joining the graft to the coronary artery must be addressed. The angled anastomotic junction created by the connector embodiments of the invention accommodate this mis-match in ratio between the host vessel and graft inner diameters. In fact the angled design enables the connector embodiments to address any ratio between graft and host vessel inner diameters.

The present invention is adapted to handle these issues as well as others as may be apparent to those with skill in the art. The angled-type connectors described herein may be employed with precision and speed, resulting in treatment efficacy not heretofore possible.

The ability to convert coronary artery bypass grafting procedures and peripheral bypass grafting procedures to less invasive approaches involving small incisions and remote creation of anastomoses are particularly difficult with conventional suturing techniques and are amenable to the embodiments and approaches for the angled connectors and associated components.

### SUMMARY OF THE INVENTION

The invention includes various improvements in end-side anastomosis systems. Particularly, connectors for producing distal anatomoses are described. They each include a fitting comprising a heel section with a trailing segment that is deflectable about a hinge region to allow for placement and securing the device. Curvilinear side and forward-facing portions are preferred. Most preferably, these portions are configured to conform to the shape of a host vessel and direct the opening (incision) through the host vessel to assume the shape defined by the fitting. Such a fitting may alone serve as a connector between a host vessel and a graft provided that it includes features capable of compressing the host vessel and graft in place or otherwise maintaining close apposition between the graft and host vessel. Alternatively, the connector may comprise a fitting in combination with a collar adapted to secure a graft to the fitting and compress the graft and host vessel.

Various features for improving the deployability of a connector, hemostasis at the connector to host vessel interface, and blood flow through the anastomoses may be provided by the invention. Further, various tools for use in preparing for and creating an end-side anastomosis may comprise part of the invention. Finally, various instruments and accessories decreasing the access required to deploy the connector to enable minimally invasive surgical approaches may comprise part of the invention.

While connectors and deployment devices according to the present invention are preferably used in peripheral and coronary artery bypass grafting procedures, at a distal (out-flow) or proximal (in-flow) location, it is to be understood that the systems described herein may be used for purposes other than creating artery-to-artery or vein-to-artery anastomoses. The systems may also be used to produce anastomoses between bypass grafts and host vessels to treat other occlusions, vascular abnormalities such as stenoses, thromboses, aneurysms, fistulas and other indications requiring a bypass graft. The system of the present invention is also useful in bypassing stented vessels that have restenosed, and saphenous vein bypass grafts that have thrombosed or stenosed. Further, the invention may have other applications, such as producing arterial to venous shunts or fistulas for hemodialysis, bypassing lesions and scar tissue located in the fallopian tubes causing infertility, attaching the ureter to the kidneys during transplants, and treating gastrointestinal defects (e.g., occlusions, ulcers, obstructions, etc.).

The present invention variously includes the devices as well as the methodology disclosed. Furthermore, it is contemplated that sub-combinations of features, especially of the connector features disclosed, comprise aspects of the invention.

### BRIEF DESCRIPTION OF THE DRAWINGS

Each of the following figures diagrammatically illustrates aspects of the present invention. The illustrations provide examples of the invention described herein. Like elements in the various figures often are represented by identical numbering. For the sake of clarity, some such numbering may be omitted.

FIG. 1 shows a side view of an installed connector with a collar that secures a graft to the fitting and affixes the connector and graft assembly to a vessel wall.

FIG. 2 shows a side-sectional view of the installed connector in FIG. 1.

FIGS. 3a and 3b show side and isometric views of a formed fitting as may be used according to that shown in FIGS. 1 and 2.

FIGS. 4a and 4b show side and top views of a formed collar as may be used according to that shown in FIGS. 1 and 2.

FIGS. 5a and 5b-show side and top views of the collar in FIGS. 4a and b deflected using an external force during deployment.

FIGS. 6a and 6b show bottom views of two fitting embodiments thermally formed to accommodate different graft to host vessel inner diameter ratios.

FIGS. 6c and 6d show bottom views of two collar embodiments, along with the fitting embodiments in FIGS. 6a and 6b, that accommodate different graft to host vessel inner diameter ratios.

FIGS. 7a and 7b show top and side views of an alternative formed fitting embodiment that locates the toe flap of the graft against the interior surface of the host vessel.

FIGS. 8a and 8b show top and side views of a formed collar embodiment that cooperates with the fitting embodiment in FIGS. 7a and 7b to secure a graft to a host vessel.

FIGS. 9a and 9b show a single-piece connector embodiment.

FIG. 9c shows the hinge locations of the connector in FIGS. 9a and 9b.

FIGS. 9d and 9e show top and side views of the connector in FIGS. 9a and 9b with a graft secured.

FIGS. 10a and 10b show the components of a loading tool used to secure a graft between a fitting and a collar.

FIG. 10c shows a perspective view of a loading tool base for use in securing a graft to the fitting and collar.

FIG. 10d shows a perspective view of a pushing tool for use with the loading tool base of FIG. 10c.

FIGS. 11a, 11b, and 11c show a side view, an end view, and a bottom view of an alternative inner frame (fitting) cartridge component of a loading tool embodiment.

FIGS. 12a to 12d show an outer frame (collar) cartridge component of a loading tool embodiment.

FIG. 13a is a perspective view of a loading platform of the present invention. FIG. 13b shows an exploded view of the components of the loading platform of FIG. 13a in operative relationship with the inner frame cartridge of FIGS. 11a to 11c and the outer frame cartridge of FIGS. 12a to 12d.

FIGS. 14a and 14b show an exploded view and a detailed view of a deployment tool embodiment.

FIGS. 14c and 14d show side-sectional views of the deployment tool embodiment in FIGS. 14a and 14b.

FIGS. 15a and 15b show side views of the deflecting mechanisms of the deployment tool embodiment in FIGS. 14a to 14d in the released state and deflected state respectively.

FIGS. 16a and 16b show a perspective view and an end view of a repositioning tool.

FIGS. 17a and 17b show a perspective view and an end view of a removal/repositioning tool.

FIGS. 18a and 18b show a perspective view and an end view of a removal tool.

FIGS. 19a and 19b illustrate another removal tool of the present invention in open and closed positions, respectively.

FIG. 20a illustrates another removal tool. FIGS. 20b and 20c are enlarged side and top views, respectively, of the end effector of the removal tool of FIG. 20a.

FIG. 21a shows a perspective view of a connector-to-graft loading system of the present invention. FIG. 21b is a perspective view of a mating cartridge of the loading system of FIG. 21a. FIG. 21c the mating cartridge of FIG. 21b in use with a deployment tool of the present invention.

FIGS. 22a, 22b and 22c show exposed side views of end effectors of other deployment tools of the present invention.

FIGS. 23a and 23b shows views of the proximal end of a deployment tool of the present invention having an axial advancement adjustment knob.

FIG. 24 illustrates a perspective view of a visualization and access tool of the present invention in use.

FIGS. 25a-25e illustrate various embodiments of visualization/access tools of the present invention.

FIGS. 26a and 26b illustrate exemplary cross-sectional shapes of the end effectors of the visualization tools.

FIGS. 27a and 27b illustrate perspective and side views, respectively, of a suction-based visualization/access tool of the present invention.

FIGS. 28a and 28b illustrate perspective and side views, respectively, of another suction-based visualization/access tool of the present invention.

FIGS. 29a-29d illustrate another embodiment of a connector of the present invention.

FIGS. 30a-30d illustrate another embodiment of a connector of the present invention.

FIGS. 31a-31c illustrate a partial exploded view of a deployment tool of the present invention.

FIGS. 32a and 32b illustrate another device for deploying and removing a connector.

### DETAILED DESCRIPTION OF THE INVENTION

The variations of the invention discussed herein are applicable to robotic surgery, endoscopic, and other less invasive (i.e., minimally invasive) surgery. As noted above, the present invention includes variations of anastomosis connectors having features adapted to perform angled anastomoses. Anastomotic connectors, tools and associated methodology for producing in-flow (proximal) and out-flow (distal) anastomoses are described variously in U.S. and foreign patent and applications entitled, “Percutaneous Bypass Graft and Securing System”, U.S. Pat. No. 5,989,276; “Percutaneous Bypass Graft and Securing System”, U.S. Pat. No. 6,293,955; “Percutaneous Bypass Graft Securing System”, PCT Publication No. WO 98/19625; “Sutureless Anastomosis Systems”, U.S. patent application Ser. No. 09/329,503; “Sutureless Anastomosis Systems”, PCT Publication No. WO 99/65409; “Thermal Securing Anastomosis Systems” U.S. Pat. No. 6,361,559; “Thermal Securing Anastomosis Systems”, PCT Publication No. WO 99/63910; “Aortic Aneurysm Treatment Sytems”, U.S. patent application Ser. No. 09/329,658; “Aortic Aneurysm Treatment Systems”, PCT Publication No. WO 00/15144; “Additional Sutureless Anastomosis Embodiments”, U.S. patent application Ser. No. 09/654,216; “Anastomosis Systems”, U.S. patent application Ser. No. 09/730,366; “End-Side Anastomosis Systems”, PCT Publication No. WO 01/416653; “Advanced Anastomosis Systems”, U.S. patent application Ser. No. 09/770,560; “Distal Anastomosis System”, U.S. patent application Ser. No. 09/899,346; “Distal Anastomosis System”, U.S. patent application Ser. No. 09/991,469; “Improved Distal Anastomosis System”, U.S. Provisional Application Ser. No. 60/333,276; and “Sutureless Anastomosis System Deployment Concepts”, U.S. patent application Ser. No. 09/927,978 and applications and patents claiming benefit hereto, all commonly owned by Converge Medical, Inc. and each of which is incorporated herein by reference in its entirety.

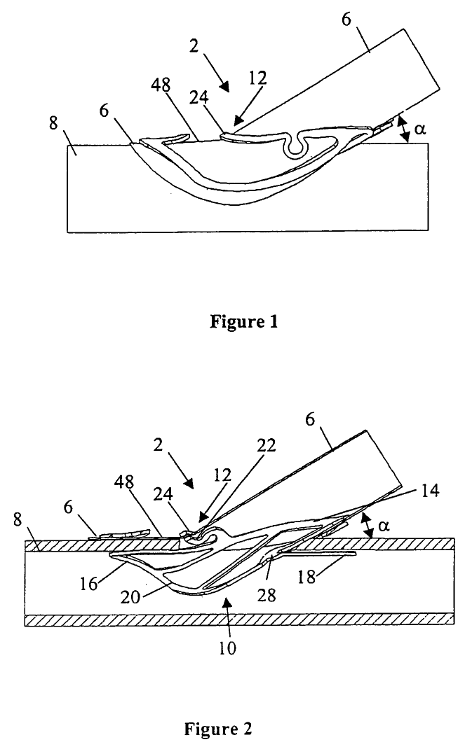

FIGS. 1 and 2 show angled anastomoses (2) formed by connectors (4) according to the present invention. Each connector (4) attaches a graft (6) to a host vessel (8). The host vessel may be any vessel or tubular structure to which a graft or other tubular structure is secured. During Coronary Artery Bypass Grafting (CABG) surgery, the host vessel is a coronary artery (Left Anterior Descending Artery, Diagonal, Circumflex, Obtuse Marginal, Right Coronary Artery, PDA, etc.), ascending aorta, subclavian artery or other vessel capable of bypassing an obstruction or stenosis by functioning as an in-flow or out-flow anastomotic junction. During Peripheral Grafting surgery, the host vessel is a popliteal artery, femoral artery, iliac artery, the aorta, carotid artery, radial artery, renal artery, hepatic artery, mesenteric artery, cerebral artery, saphenous vein, femoral vein, or other vessel that participates in bypassing an obstruction or stenosis by functioning as an in-flow or out-flow anastomotic junction. For CABG and peripheral vascular procedures, the graft (6) comprises an autologous vessel such as a saphenous vein, radial artery, left internal mammary artery, right internal mammary artery, other tissue (e.g. pericardium, submucosal, etc.) formed into a tubular structure, a synthetic graft (such as expanded PTFE or urethane derivatives), a genetically produced vessel, a donor vessel, or other tubular structure. In addition, one anastomoses' graft may function as another anastomoses' host vessel where connector are also used as in-flow anastomotic junctions to produce a series of jumper connections from a main graft to several spaced apart target conduits.

Connector Embodiments & Associated Components

The connector in FIG. 1 includes a fitting (hidden) secured to the graft and the host vessel with a collar (12). FIG. 2 shows a side-sectional view of the connector in FIG. 1. The connector in FIGS. 1 and 2 may be utilized as an out-flow anastomotic junction where blood passes through the graft, past the connector, and into the host vessel where it is capable of flowing antegrade and retrograde. Alternatively, the connector in FIGS. 1 and 2 may be utilized as an in-flow anastomotic junction where blood passed through the host vessel, past the connector, and into the graft.

Referring to FIG. 2, various features of fitting (10) may be observed. First, it is noted that fitting and attached graft (6) are preferably configured so its base or body (14) is at an angle α with respect to host vessel (8). Connectors (2) are shown at approximately a 30° angle. Preferred angles for distal anastomosis range from about 20° to about 70°. A more preferable range is from about 25° to about 45°. Most preferably, they are approximately 28-30°. Because of the design of the connector, the angle helps maintain hemostasis and optimize blood flow once the anastomosis is created and retracted organs and tissue bear upon the site. Pressure created by such action will not dislodge connector (4) or kink or collapse graft (6) since the connector allows the graft (6) to extend at an acute angle such that the graft closely apposes the host vessel, and lies substantially in line with the host vessel and adjacent anatomy. In addition to improving blood-carry capability of the conduit in assuring stability of the connector, including some angle in the connector enables the manner of deployment and attachment taught below.

As shown in FIGS. 2, 3a, 3b, 29a, 29b, 30a and 30b fitting (10) includes at least a front or leading segment (16) and a rear or trailing segment (18). When situated to form an anastomosis, these segments lie approximately in line with host vessel (8). So-placed, they prevent removal of the connector from the host vessel. Optional lateral or side portions (20) may also aid in this regard. This is especially the case when forming an anastomosis with a very small diameter vessel (such as a 1 to 4 mm inner diameter host vessel). Furthermore, lateral portions (20) extend beyond the plane of the trailing segment (18) and interconnect with the leading segment (16) to ensure the host vessel tissue about the opening through the host vessel is completely captured around the anastomosis thereby ensuring a physical barrier to leakage. This may be true irrespective of the size of host vessel (8). The one or more lateral portions (20) on each side of fitting (10) also provide a smooth transition between the leading and trailing portions of fitting (10) to facilitate insertion of the connector through an opening in the host vessel and help moderate or alleviate trauma to the interior of the host vessel (8) while deploying the connector.

A lateral portion may be provided integrally with a form providing at least part of leading segment (16) and trailing segment (18). As described above, this continuous coverage ensures complete tissue capture between the fitting (10) inside the host vessel and the collar (not shown) outside the host vessel. Complete coverage ensures hemostasis at the vessel to graft interface.

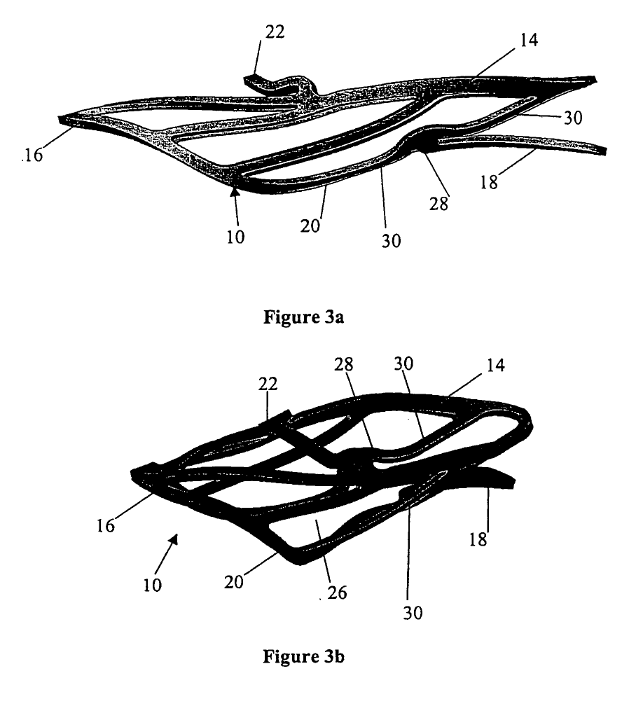

As shown in FIGS. 3a, 3b, 29a-29d and 30a-30d additional optional features of fitting (10) include tabs or latches (22) to assist in securing graft (6) and/or optional collar (12). Such tabs may be oriented to grip graft (6) as shown in FIG. 2. One or more tabs may also be adapted to form a locking interface with one or more complementary tabs or latches (24) optionally included in collar (12). Also, the height or amount of material incorporated in the base of the fitting may be varied. In order to utilize as little material as possible to join the various segments, base (14) may be provided by a narrow band of material as shown in FIG. 3a, 7b, 29a, 29b, 30a and 30b or otherwise. To achieve proper relative placement of these features, base (14) may be curved or undulate.

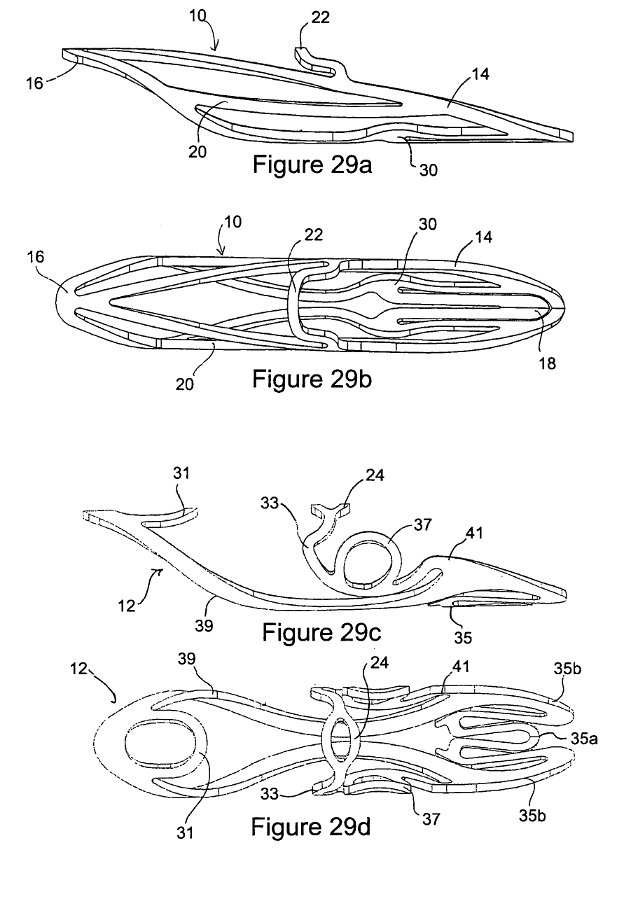

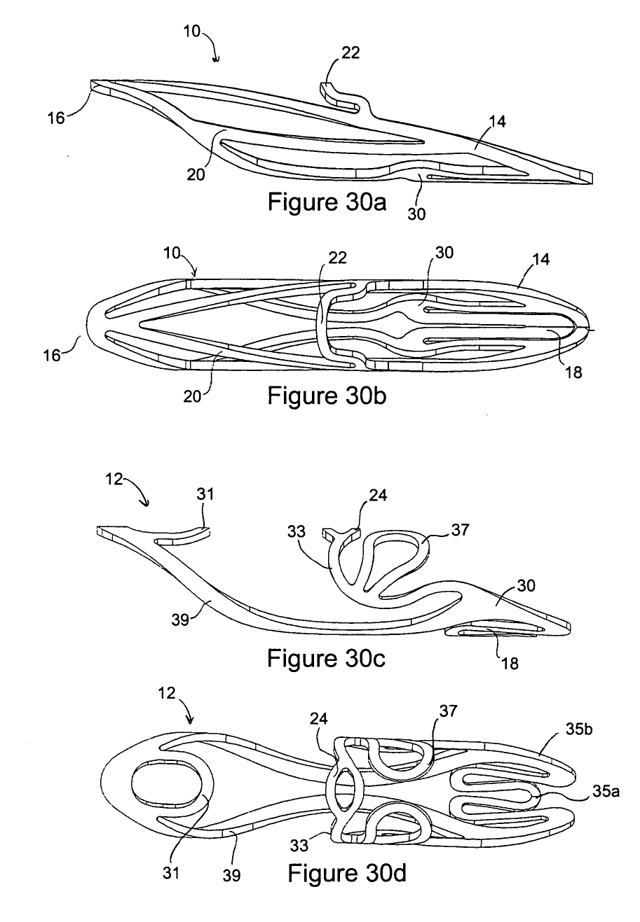

The connectors of FIGS. 29a-29d and 30a-30b include fittings or inner frames (10) and collars or outer frames (12), respectively, having features similar to those described above. These connectors differs, however, in that ears (37) of outer frame (12) are directed upward rather than downward, and as such, further ensure that gauze, or other hospital device does not catch on the ears (37), which could cause dislodgement of the coupler if the gauze or other hospital device is placed into engagement with the ears (37) and pulled upward with substantial force. Another feature of these connectors is the extended length of the leading segment (16) of inner frame (10) and of the leading portion of outer frame (12). The extended length from the distal end of the leading segment (16) to the distal end of the tab or latch (22) enables using a decreased arteriotomy length when inserting the leading segment and deflected trailing segment through the arteriotomy, and/or facilitates visualization of the trailing segment relative to the proximal end, or heel end, of the arteriotomy during deployment. The trailing segment (18) is held in a deflected orientation during deployment such that the trailing segment extends away from its resting position from about 90 to about 145 degrees. By increasing the length of the leading segment (16) that can be inserted into the arteriotomy, the deflected trailing segment is accordingly able to be inserted further into the arteriotomy. This increases visualization of the trailing segment relative to the heel aspect of the arteriotomy or enables decreasing the trailing segment deflection angle to below 90 degrees from its resting orientation; both options increase visualization of the trailing segment relative to the heel aspect of the arteriotomy thereby ensuring successful deployment of the coupler into the host vessel.

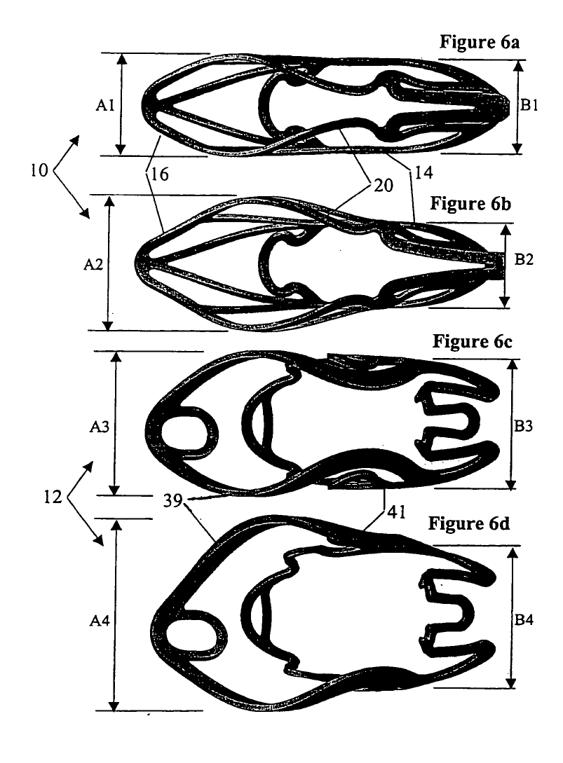

As shown in FIG. 3b, the connector opening (26) may have an ovalized or elliptical opening to the anastomosis, or may have a circular bore. As will be discussed below, the connector is preferably fabricated from a raw tube that is laser cut into the desired pattern and thermally formed into the desired resting configuration as shown in FIGS. 3a and 3b. This inherent profile may be altered by closing the width between opposite sides of the lateral portions (20) and/or base (14) causing the connector to assume an ovalized profile with the major axis extending from the leading segment (16) towards the trailing segment (18) and the minor axis perpendicular to the major axis, as shown in FIG. 6a. Configuring fitting (10) with an ovalized opening (26) may be useful in providing an interface to a smaller host vessel. As shown in FIG. 6a, ovalizing the profile at the lateral portions (20) to a width, A1, while maintaining the profile of the base (14) to a width, B1, provides a manner in which to account for the optimal transition in the size difference between a smaller diameter host vessel and what is often a larger diameter opening of the graft by transitioning the geometry change from the ovalized anastomotic junction cross-section to the more circular graft cross-section. In this case A1≦B1. For example, a 30°, 3 mm connector having B1=0.117″ and A1=0.110″ is capable of transitioning a graft with an inner diameter from 3 mm to 5 mm to a host vessel with an inner diameter from 2 mm to 4 mm. A 30°, 3 mm connector having B1=0.117″ and A1=0.080″ is capable of transitioning a graft with an inner diameter from 3 mm to 5 mm to a host vessel with an inner diameter from 1.25 mm to 2.5 mm. The ovalization increases the available perimeter to accommodate a host vessel without having to alter the diameter of the connector. Instead, a connector is lengthened by ovalizing its opening to accommodate smaller host vessels without having to change the diameter of the base and/or graft. Ovalizing the connector is an acceptable alteration in connector geometry since only the size of the arteriotomy made in the host vessel need be lengthened to fit the connector in place.

The angled connector geometry provides a further enhancement in that a single version accommodates a wide range of graft diameters. By angling the graft relative to the host vessel, the cut end of the graft, which defines the graft toe (48) and the angle the graft extends from the connector may be modified to produce a cross-section that matches the specific connector size.

As shown in FIG. 6b, the separation between the lateral portions (20) of the fitting (10) may be increased, A2, such that it exceeds the diameter, B2, of the base (14) to enable transitioning a larger diameter host vessel to a smaller diameter graft. This is particularly relevant when using the angled connector as an in-flow anastomotic junction between a large vessel (such as the aorta, iliac, subclavian, carotid artery, femoral artery, or other supplying vessel) and a smaller diameter graft.

As shown in FIGS. 6c and 6d, the collar profile matches that of the fitting to accommodate for the disparity in size between the host vessel and graft, if any. The collar of FIG. 6c matches the profile of the fitting embodiment in FIG. 6a such that A3≦B3 to apply compression against the host vessel and graft when the host vessel diameter is equal to or smaller than the diameter of the graft. Similarly, the collar of FIG. 6d matches the profile of the fitting embodiment in FIG. 6b such that A4>B4 to accommodate larger host vessel diameters compared to the graft.

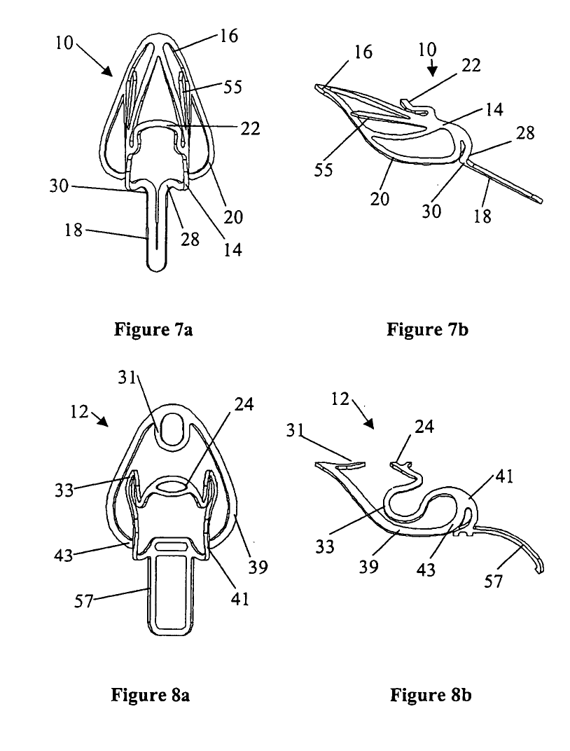

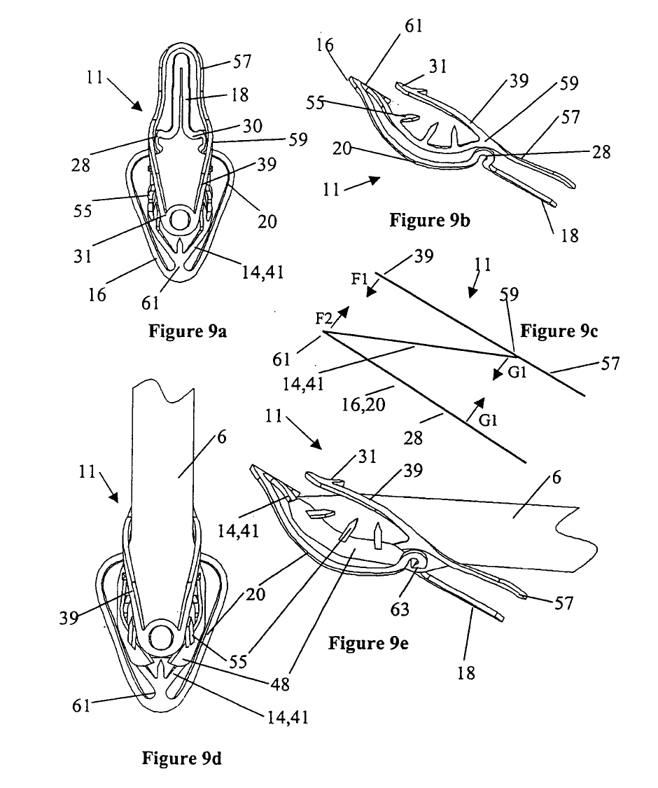

Features that are preferable for fitting (10), in addition to the basic leading and trailing segment configuration, are found in connection with a hinge section (28), shown in FIGS. 2, 3a, 3b, 7a, 7b, 9a, and 9b. Hinge section (28) may be provided in a number of configurations. However, the configurations serve the same purpose. Each of the variations shown and described allow trailing segment (18) to be displaced sufficiently to clear the host vessel wall for insertion of the connector into the host vessel by significant torsional deflection of areas between trailing segment (18) and fitting body (14). In the fitting variations shown in FIGS. 2, 3a and 3b, a pair of torsion sections (30) are presented on each side of trailing segment (18). In the fitting variations shown in FIGS. 7a, 7b, 9a, and 9b, a single torsional section (30) is presented on each side of trailing segment (18).

To displace trailing segment (18) sufficiently, the rotation about torsional sections accounts for a substantial amount of the displacement required of trailing segment (18). The additional displacement arises from bending of the trailing segment (18) relative to the junction between the trailing segment and the torsional sections.

Such dual action provides for certain advantages; namely, upon forward deflection of trailing segment (18), i.e., deflection of trailing segment (18) towards leading segment (16), the lateral portions connected to torsional sections are caused to be drawn or flexed inward. This action facilitates introduction of connector (4) into host vessel (8) by clearing portions that could otherwise interfere with entry. In addition, the design of the embodiment in FIGS. 2, 3a and 3b has a pair of torsional sections on each side of the trailing section, one integrated with the base (14) and an opposite extending one integrated with the leading section (16). The embodiment in FIGS. 2, 3a and 3b has the trailing section (18) cut from the base (14) and deflected approximately 30 degrees in its resting configuration. As such the trailing section (18) is integrated with both the base and the leading section to provide a continuous band of support throughout the anastomosis along the interior surface of the host vessel, increase the resistance to deflection once the connector is deployed, and provide a wedge between the trailing section (18) and the base (14) capable of increasing the compression forces that the trailing section (18) and the base (14) exert against the graft and the host vessel to ensure hemostasis at the heel of the anastomosis.

The embodiment in FIGS. 7a, 7b, 9a, and 9b similarly has the trailing section (18) cut from the direction of the base (14) however, the base in this embodiment has been shortened and extends from just adjacent to the trailing segment (18) to the leading segment (16). Therefore, the trailing section (18) still provides a continuous band of support throughout the anastomosis but the base does not inhibit the ability to extend the graft at a more acute angle than 28 to 30 degrees.

Turning now to the features of collar (12), FIGS. 1, 4a, 4b, 5a, 5b, 29c, 29d, 30c and 30d illustrate desirable features of this part of connector (4). One purpose of collar (12) is to secure the graft (6) and host vessel to fitting (4) and ensure the graft produces a gasket against the host vessel throughout the periphery of the anastomosis to ensure hemostasis. As noted above, optional collar tab(s) or latch(es) (24) may assist in this regard by interfacing with optional fitting tab(s) or latch(es) (22). Also, collar (12) may be resiliently biased against graft (6) and host vessel to hold it to fitting (10). Further, expansion spring members (35) may be provided to enable expanding the diameter of the collar for placement around the fitting and returning the collar towards its preformed configuration once positioned to ensure a secure fit of collar (12) about fitting (6).

The expansion spring members (35) in the embodiment in FIGS. 1, 4a, 4b, 5a, 5b, 29c, 29d, 30c and 30d incorporate a vertical undulating pattern, which enables deflection along the long outer links as the outer frame or collar is expanded from its resting diameter towards an enlarged geometry. This expansion spring (35) configuration has a middle undulation (35a) and two side undulations (35b). The length of the middle undulation is shorter than that of the side undulations (approximately ½ to ¼ shorter), and the widths and wall thicknesses are the same so enlarging the expansion spring first separates the side undulations without altering the middle undulation and only after substantial enlargement of the side undulations does the middle undulation separate. This helps orient the trailing segment (18) of the fitting (4) relative to the expansion spring (35) while loading the fitting and graft to the collar. Another alignment feature shown in FIGS. 6c and 6d are short protrusions (34) extending from the junction between the side undulations and the middle undulation that orients the trailing segment (18) relative to the expansion spring (35) and maintains that orientation during manipulation of the connector.

This expansion spring embodiment also enables lengthening the distance from the tab or latch (24) of the collar and the location on the expansion spring to which the trailing segment of the fitting abuts. As the inner frame is advanced relative to the outer frame, the trailing segment contacts the expansion spring and further force causes the lateral (outer) links of the expansion spring to deflect causing the inner frame tab or latch to move past the latch of the outer frame. This facilitates securing the collar to the fitting around the graft by locating the tab (24) of the collar beyond the tab (22) of the fitting without having to engage and dramatically pull tab (24) past the tab (22). Upon releasing the external force deflecting the collar, the expanding spring members recoil towards the undulating pattern urging the collar towards its resting, smaller diameter configuration thereby engaging the tab (24) of the collar to the tab (22) of the fitting and compressing the collar against the base (14) of the fitting.

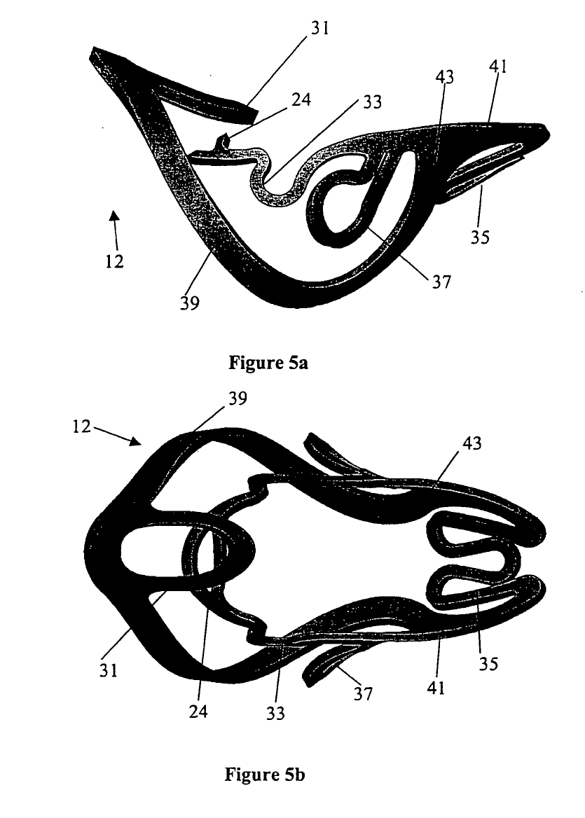

Preferably, the distal band (39) of the collar (12) extends completely around the anastomosis from the heel to the toe to overlap or interface with corresponding lateral features (20) of a complimentary fitting (10) to form a complete seal at an anastomosis site. Likewise, the shape of the bore of the collar as shown in FIGS. 6c and 6d should complement that of the fitting (e.g. FIGS. 6a and 6b respectively). In instances where the fitting has a circular bore (26), at least a mating portion of collar (12) should be substantially circular as well. In instances where fitting bore (26) is ovalized, a corresponding shape should be utilized in collar (12). For instances where the fitting is tapered in geometry from a circular profile at the graft to an ovalized or enlarged profile at the anastomotic junction, the collar should also possess such features. The distal band (39) is secured to the base of the collar at the heel to enable deflecting the distal band (39) upward during deployment, as shown in FIG. 5a. The semicircular nature of the distal band (39) causes the distal band to buckle outward as it is deflected with a deployment tool, as shown in FIG. 5b. This provides separation between the distal band (39) and the lateral sections (20) of the fitting to ensure host vessel tissue can enter this gap such that once positioned, the distal band may be released thereby compressing the graft and the host vessel against the fitting's leading section and lateral sections ensuring complete hemostasis around the periphery of the anastomosis.

Another feature of the collar (12) embodiment shown in FIGS. 1, 4a, 4b, 5a, 5b, 29c, 29d, 30c and 30d involves side spring loops (33). These side spring loops (33) enable axial extension of the tab (24) during loading of the collar over the graft and the fitting to enable placing the tab (24) of the collar into engagement with the tab (22) of the fitting without requiring significant manipulation of the fitting and collar. The utility of the side spring loops (33) is diminished if the expansion spring enables adequate lengthening of the tab (24) relative to the expansion spring yet provides additional axial lengthening of this dimension during loading of the graft and fitting to the collar.

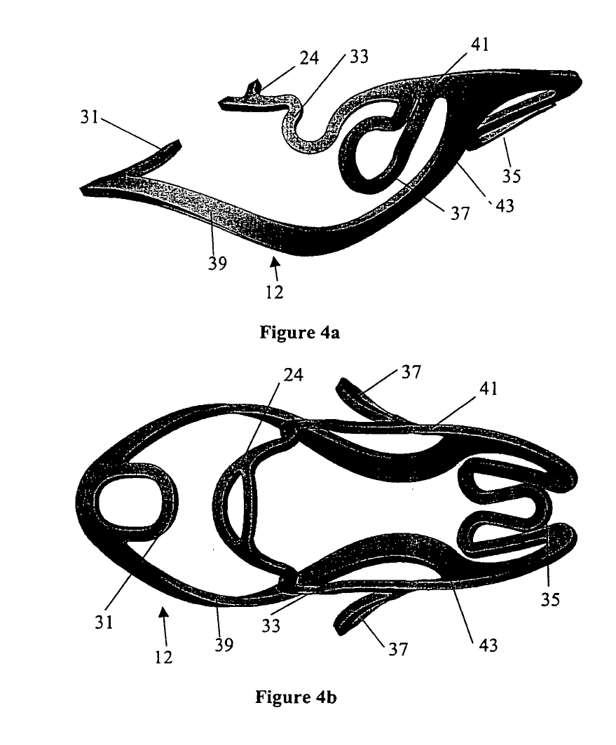

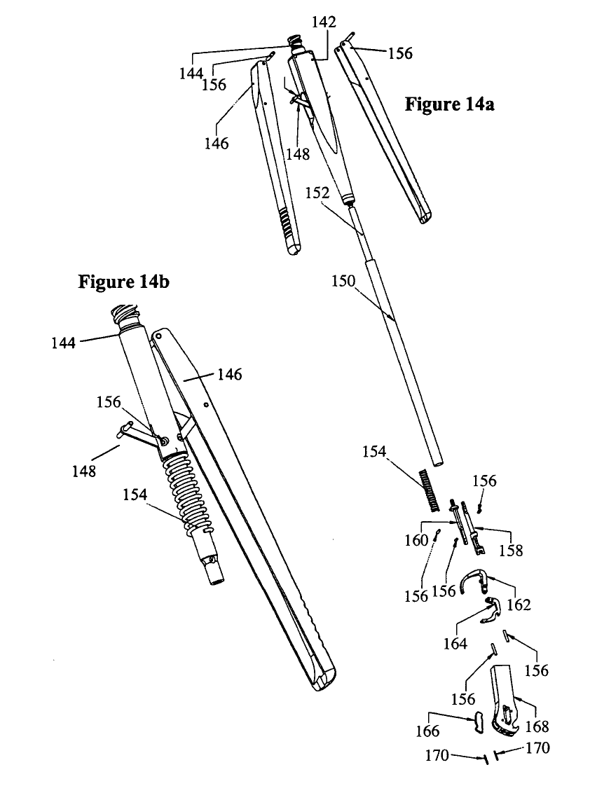

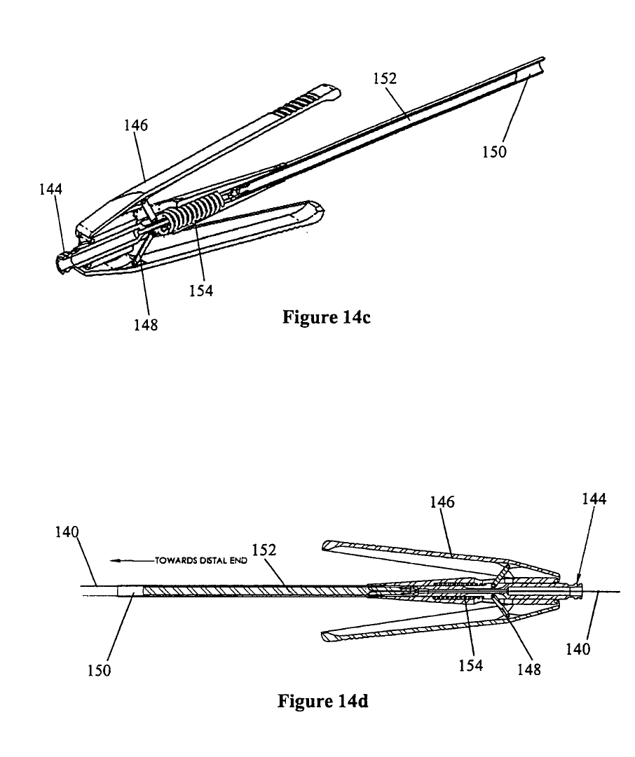

Ears (37), shown in FIGS. 4a, 4b, 5a, 5b, 29c, 29d, 30c and 30d provide an engagement point for pins of a deployment tool (discussed below with respect to FIGS. 14a-14d) to stabilize the connector during deployment or a loading tool to manipulate the collar during placement of the graft and/or locking of the fitting to the collar. The ears may or may not be thermally formed in a radially outward configuration such that the deployment tool and/or loading tool pins may be readily inserted from the top, front, or rear, depending on the location of the pins on the deployment tool.

The collar embodiments in FIGS. 4a, 4b, 5a, 5b, 29c, 29d, 30c and 30d also incorporate a grasping loop or link (31) that provides an exposed edge which the deployment tool may engage and deflect the distal band (39) relative to the base of the collar. This facilitates engagement and removal of the deployment tool relative to the collar.

Whether prepared in connection with a collar or not, connector (4) is preferably installed at an anastomosis site as shown in FIG. 2. Here, it may be observed that graft toe (48) preferably overlaps host vessel (8). A heel portion (62) may abut, overlap host vessel (8) or leave a slight gap.

When connecting a graft to a small diameter host vessel, the graft toe (48) preferably resides along the exterior surface of the host vessel so it doesn't substantially reduce the cross-sectional area of the host vessel. When a connector is provided with a collar (12), the visible result will resemble that in FIG. 1. Still, one preferred relation of graft (6) to host vessel (8) remains similar to that shown in FIG. 2, depending on the fitting configuration selected. Alternatively, the graft toe (48) may be oriented such that it resides along the interior surface of the host vessel and the host vessel overlaps the graft toe. This is an especially suitable alternative when the connector is attaching a graft to a larger diameter host vessel.

The function of the connector (as an in-flow anastomotic junction or out-flow anastomotic junction) also impacts the location of the graft toe (48) (e.g. inside the host vessel, and/or outside the host vessel). Other aspects of the anastomotic junction also impact the location of the graft toe. For example, when securing a graft to a host vessel having a large wall thickness (e.g. aorta), the graft toe (48) is preferably located along the interior surface of the host vessel so the thick cut end of the aorta is not exposed to blood flow. As such, flow disruptions are avoided by ensuring a smooth transition from the graft to the host vessel. When everting the tissue to minimize the metal exposed to blood, the graft toe is preferably located along the interior surface of the host vessel therefore the cut end of the graft and host vessel are isolated from blood flow. As shown in FIGS. 9d and 9e, the cut/beveled end of the graft toe readily everts around the toe of the fitting (10); the cut bevel easily wraps around the slightly curved cross-section of the leading segment (16) by taking opposite free edges of the cut tissue and pulling them around opposite sides of the leading segment and securing them in place by use of pins (55) and/or compressing them between two components as shown in FIGS. 9c and 9d. On the contrary, the side of a host vessel is extremely difficult to evert because all edges of the tissue are constrained so the only way to evert is to over-stretch the tissue which results in unwanted damage.

FIGS. 7a and 7b show an alternative fitting embodiment (10) that along with collar embodiment shown in FIGS. 8a and 8b produce a connector capable of producing an in-flow anastomotic junction and/or an anastomosis having a host vessel to graft inner diameter ratio>>1. As shown in FIG. 7a, the separation between lateral portions (20) is increased to accommodate the larger host vessel while the separation between the sides of the base (14) accommodate the smaller graft. As described above, a latch or tab (22) on the fitting mates with the corresponding latch or tab (24) on the collar (see FIGS. 7a and 8a). The trailing segment (18) in this embodiment is designed to penetrate through a small puncture in the heel portion of the graft just proximal to the end of the incision (described below). This secures the heel of the graft to this fitting embodiment because the stem region at the heel of the fitting is non-existent. Pins (55) may be used to hold the toe region of the graft against the fitting during insertion through the arteriotomy ensuring the graft toe region resides against the interior surface of the host vessel. The collar incorporates a heel segment (57) to account for the elimination of the wedge with this embodiment. A slot in the heel region accommodates insertion of the trailing segment (18) to lock the collar to the fitting at the heel. As previously stated, tab (24) may be locked to tab (22). Side springs (33) enable extension of tab (24) beyond tab (22) during loading and return towards its resting configuration when the external, extension force is removed thereby locking tab (24) to tab (22). A distal band (39) matches the leading segment (16) and lateral portions (20) of the fitting to provide compression around the anastomosis. A grasping loop (31) enables deflecting the distal band (39) as will be described below. It should be noted that this embodiment may be modified to accommodate host vessel to graft inner diameters ≦1 by thermally forming the lateral portions (20) of fitting and separation of distal band (39) of collar to accommodate host vessel inner diameters smaller than or equal to the graft inner diameter.

FIGS. 9a and 9b provide an all-in-one connector embodiment that incorporates the fitting and collar functions into a unitary connector. This unitary connector (11) incorporates a leading segment (16) that defines lateral portions (20) which are integrated to a trailing segment (18). As described in FIGS. 7a and 7b above and shown in FIG. 9e, the trailing segment (18) is placed through a puncture (63) in the heel of the graft just beyond the incision through the graft that produces the graft toe. This locks the graft to the connector at the heel region. Leading segment (16) produces a hinge (61) to base (14, 41) that enables deflecting the leading segment, lateral portions, and trailing segment while placing the graft toe between the lateral portions (20) and base (14, 41). Once positioned, the external force deflecting the lateral portions is removed allowing the lateral portions to return towards their preformed shape compressing the graft toe (48) between the lateral portions (20) and the base (14, 41). A second hinge (59) integrates the distal band (39) and the heel segment (57) to the base (14, 41). The distal band (39) is deflected during deployment, as described below, to provide a separation that host vessel tissue may enter for compressing the graft and host vessel between components of the connector. The heel segment (57) compresses the host vessel against the trailing segment (18) to maintain position of the connector in the host vessel and stabilizes the graft at the heel of the anastomosis. Pins (55) may be used to evert the graft toe (48) to lock the graft in place. The pins (55) may be used when the compression force between the lateral portions (20) and the base (14,41) about hinge (61) is not adequate to lock the graft to the connector or when the operator wants to isolate the cut end of the graft from blood flow. FIGS. 9d and 9e show the unitary connector (11) with a graft toe (48) clamped between the lateral portions (20) and the base (14,41) and everted over pins (55). FIG. 9c shows the compression forces used to lock the graft and host vessel to the unitary connector. Forces (F1, F2, G1, and G2) may be optimized by altering the stiffness and/or spring constants of hinges (61 and 59) to ensure that the graft and host vessel are captured by and locked to the unitary connector (11).

Angled Anastomosis Procedure and Accessory Devices

Now that many of the device features of the invention have been described, the process associated therewith is set forth in the order in which it is preferred that a surgeon or surgical team take action to perform a coronary bypass procedure, peripheral bypass procedure, or other procedure associated with creating anastomoses between tubular body structures during surgical, minimally invasive, endoscopic, robotic, catheter-based, or a combination of these approaches. Variation of this procedure is, of course, contemplated. Furthermore, it is to be understood that the devices described herein may be used outside of this context.

This being said, after opening a patient and taking a measurement between intended target sites for in-flow (proximal) and out-flow (distal) anastomoses, a graft member (6) of sufficient length is obtained. Typically this will be a saphenous vein. Alternately, another harvested vessel (such as the left internal mammary artery, right internal mammary artery, radial artery, or other autologous vessel), a synthetic graft (e.g. ePTFE, urethane, etc.), non-vascular autologous tissue (e.g. pericardium, submucosa, etc.), a genetically engineered tubular structure, or a donor tissue may be used as a graft.

Especially in the case where an organic member is used, the vessel will be sized to determine the appropriate connector size. This is preferably done with reference to the inner diameter of the graft by inserting pins of increasing size (e.g. by 0.25 mm increments) until the graft no longer easily fits over a given pin. The size of the largest pin over which graft easily fits over sets the inner diameter of the graft. Alternatively, a “go/no-go” gauge may be used where a single connector covers a wide range of graft inner diameters. The “go/no-go” gauge would have a minimum inner diameter and a maximum inner diameter at which the inner diameter of the graft should reside to be used with the specific connector configuration.

Next, a connector for producing an anastomosis at a desired angle, and having an appropriate size is chosen. The size of fitting (10) and optional collar (12) covers a range of graft inner diameters and is preferably chosen by matching the first incremental size over the inner diameter of the graft to a chart of connector sizes that accommodate the measured graft diameter. It is contemplated that connector component sizes may be sized to fit grafts of a diameter from about 2 mm to about 6 mm progressively, at 0.5 mm to 2.0 mm increments. The acute angle of the connector embodiments enables a specific connector size to accommodate a wide range of graft sizes because the graft is oriented at an angle relative to the connector bore and this relationship may alter based on the size matching between the graft and the connector. For example, a 3 mm diameter connector has been demonstrated to accommodate graft inner diameters between 3 mm and 5 mm without constricting the lumen of the graft or otherwise adversely affecting the transition from the graft to the host vessel with respect to flow barriers or disruptions.

Once appropriately sized connector components are chosen, a graft is skeletonized 10 mm away from the end to be used in connection with the anastomosis. This may be accomplished by holding the adventitia tissue away from the graft with forceps and removing selected portions with Potts or Dissecting scissors.

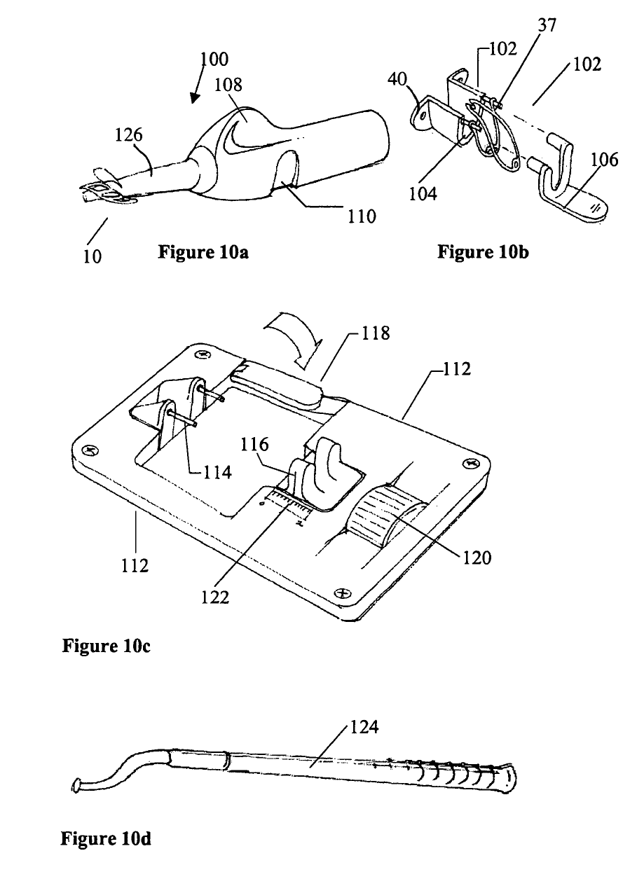

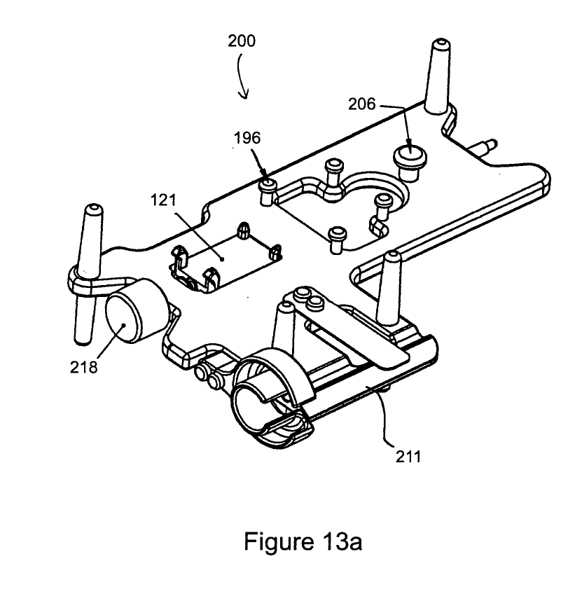

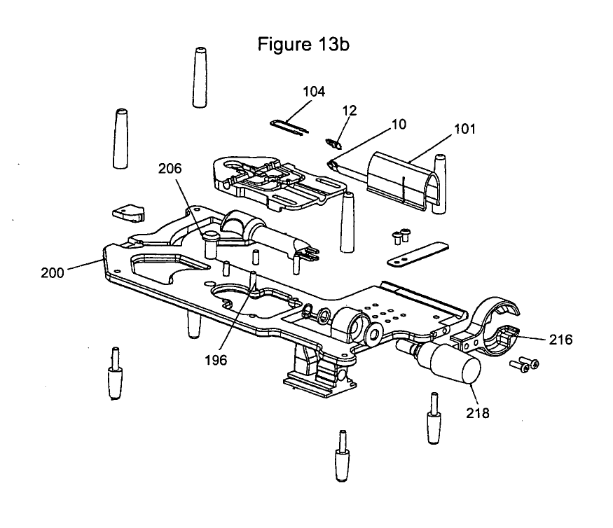

At this stage, graft (6) is passed through the collar (12), which has already been expanded to facilitate advancing the graft. The collar (12) may be housed or prepackaged on an outer frame (collar) loading cartridge (102) (see FIG. 10b) and (105) (see FIGS. 12a to 12d) which, when attached to a loading tool base (112) (see FIG. 10c) and (200) (see FIGS. 13a and 13b), may be expanded by spreading the ears (37) of the collar (12) apart thereby expanding the collar (12) at the expansion spring and providing an enlarged lumen through which to pass the graft.

As shown in FIG. 10b, the outer frame cartridge (102) may contain pins (104) that are used to stabilize the collar or outer frame (12) during shipment and expansion on the loading tool. A mating insert (106) may be used to stabilize the collar (12) relative to the outer frame cartridge (102) during shipping; this insert (106) is removed and disposed prior to placement of the outer frame cartridge within the loading tool base (112). The interlock enables the outer frame cartridge to be temporarily secured to the loading tool base (112) during placement of the graft and latching of the fitting.

Another embodiment of an outer frame cartridge (105) is illustrated in FIG. 12. Outer frame cartridge (105) has a slim, low-profile design and provides a flex region (113) that enables increasing the separation of the opposing ends of the removable clip or interlock mechanism (115) straddling flex region (113). Clip (115) has pins around which the outer frame (12) ears area looped. The interlock mechanism (115) enables the outer frame cartridge to be temporarily secured to the loading tool base or platform (discussed below) during placement of the graft and latching of the fitting and maintains the outer frame (12) in an expanded state for receiving a graft vessel. Outer frame (12) is preferably provided preloaded or attached to interlock mechanism (115) which itself is provided preloaded or mounted on outer frame cartridge (105). As the outer frame cartridge is advanced over the loading tool, the opposing flex regions (113) are urged apart causing the outer frame resting on the clip (115) to enlarge thereby increasing the diameter of the opening through the outer frame so the graft can be inserted through the outer frame without causing trauma to the graft.

In order to facilitate the insertion of the graft (6) into outer frame (12) and then the insertion of the inner frame (10) into graft, both of which require care and accuracy, a loading tool (112), as shown in FIG. 10c, may be used to stabilize outer frame cartridge (102). Outer frame cartridge (102) has mounting tabs (40) having bores for receiving pins (114) of the loading tool (112). Loading platform (200), as shown in FIG. 13a, is used with outer frame cartridge (105) of FIG. 12 and has alignment posts or guides (196) for holding outer frame cartridge (105). Loading platform (200) also includes a deployment tool holder (211) as well as features to stabilize the deployment tool (discussed in detail below) while placing the connector assembly into the deployment tool, shown in FIG. 14a.

In order to receive graft (6) through its bore, outer frame (12) is expanded. This is accomplished by means of lever (118) on the outer frame cartridge (102) of FIG. 10c. With outer frame cartridge (105) of FIG. 13a, this is accomplished automatically as the outer frame cartridge (105) is locked to the loading platform (200). The graft (6) may now be advanced through collar or outer frame (12), as illustrated in FIG. 1. This is may be accomplished with an elongate, low profile clamp or forceps to pull the graft through the expanded collar. Once the graft is positioned, an incision from the free end of the graft is created to define the graft toe (48). The length of this incision depends on the diameter of the connector and the angle of the anastomosis. For a 30°, 3 mm connector, a 9 to 10 mm incision is created to define the graft toe (48). The graft toe (48) must be sized so as to substantially cover the leading segment (16) of the fitting (10) and extend around the lateral portions (20), as shown in FIG. 2. The graft toe (48) provides the interface at which the cut edges of the host vessel (8) are clamped thereby ensuring hemostasis.

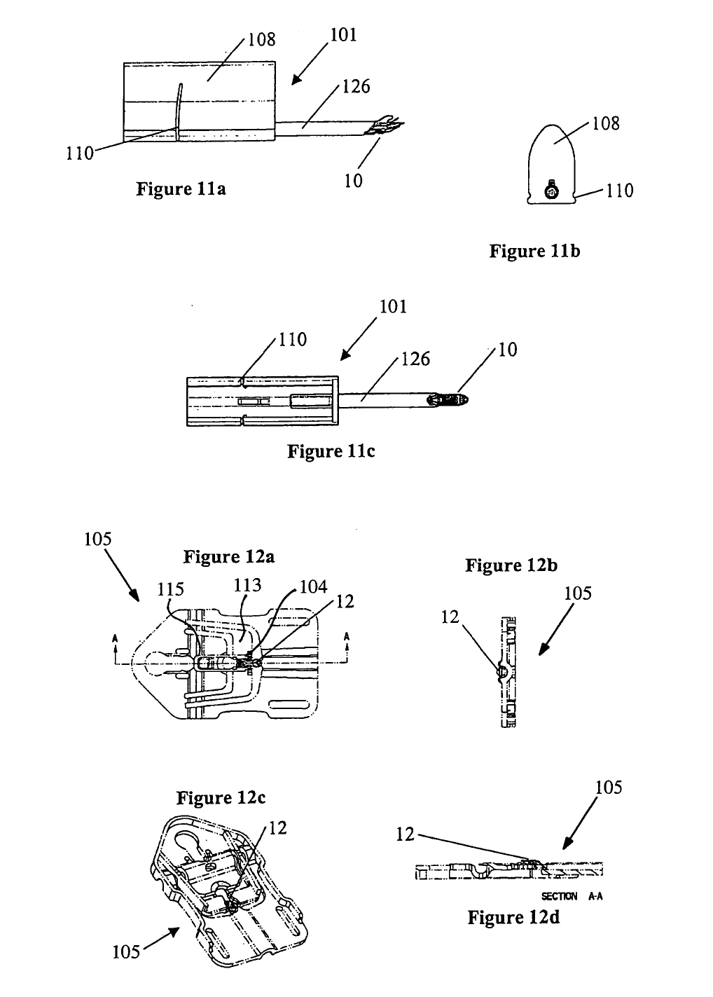

The outer frame (12) and graft (6) are now ready to receive the fitting or inner frame (10). An inner frame cartridge (100) is used to advance the outer frame (10) into the cut end of the graft such that the trailing segment (18) of the fitting is oriented into engagement with the expansion spring (35) of the collar or outer frame (12). FIGS. 10a, and 11a to 11c illustrate different variations of the inner frame cartridge (100). Inner frame cartridges (100) and (101) of FIGS. 10 and 11, respectively, includes a handle (108) and a shaft (126) extending from the handle. The distal end of shaft (126) is configured to securely hold an inner frame (10) of the present invention. Like the outer frame cartridges, the inner frame cartridges are configured to be fixedly engaged with the loading tool or platform. Inner frame cartridge (100) of FIG. 10a includes a snap or slot (110) on handle (108) which, when aligned with a dock (116) of loading tool (112), allows handle (108) to be snapped into engagement with loading tool (112). Similarly, inner frame cartridge (101) of FIG. 11 includes a slot (110) on handle (108) which defines a living hinge on the inner frame cartridge so the inner frame can fit over the sliding plate (171) of loading tool (200) of FIG. 13a and snap over and lock to the opposing catches of the inner frame plate (171).

Holding handle (108) of the inner frame cartridge, a fitting or inner frame (10), which has been operatively engaged with the distal end of shaft (126), is guided such that the base (14) of the fitting passes into the cut end of the graft (6) and under the expansion spring (35) of the collar or outer frame (12) and such that the trailing segment (18) of the fitting resides outside the graft and expansion spring. Once the trailing segment (18) is appropriately positioned, the inner frame cartridge (100) is snapped into engagement with the loading tool (112) as described above. Then the inner frame cartridge (100) is advanced using a shaft dial (120 or 218) thereby advancing the fitting relative to the collar. An indicator gauge (122) (provided on the loading tool embodiment of FIG. 10c only) indicates the distance advanced by the fitting. The expansion spring (35) of the outer frame stretches at the side undulations causing the distance between the tabs of the outer frame and the inner frame to shorten. Once the inner frame cartridge (100) is fully advanced, the tab (24) of the collar extends beyond the tab (22) of the fitting. It has been demonstrated that a 0.070″ to 0.150″ extension of the collar at the expansion spring places the tab (24) of the collar beyond the tab (22) of the fitting. The loading tool is rotated 180 degrees and a pusher (124) (see FIG. 10d) is used to apply downward pressure against the tab or latch (24) of the outer frame or collar. Simultaneously, the shaft dial (120) of the loading tool is used to retract the inner frame cartridge thereby allowing the expansion spring of outer fitting (12) to return towards its resting undulating shape meanwhile engaging the outer frame tab or latch (24) and the inner frame latch (22) with the graft locked between the two latches. In placing fitting (10) into graft (6), it is to be set in relation to collar (12) in a complementary manner. When optional tabs (22) and (24) are provided, these features can easily be used to help align a fitting and a collar relative to each other. Either way, once collar (12) and fitting (10) are properly aligned, tabs and/or locking features (36) are engaged with each other, collar (12) is released onto graft (6), and a final check is made to ensure accurate component placement and graft coverage. Once in place about fitting (18), graft (6) may be trimmed to more closely conform to the shape of the connector elements, particularly the distal band (39) of the collar (12). At this point the connector and graft assembly is complete and ready for deployment.

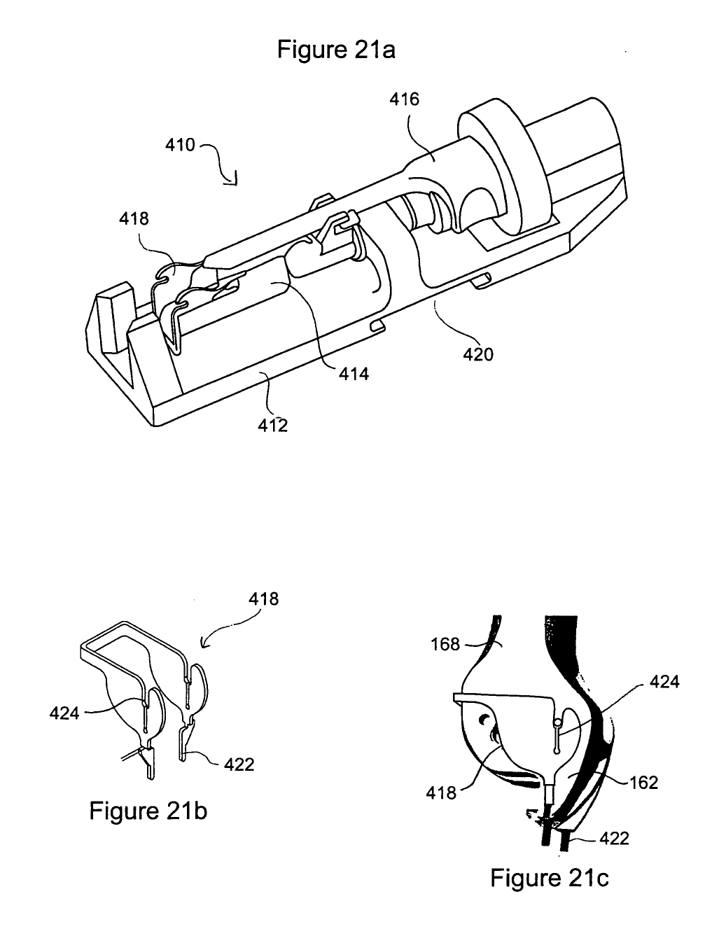

FIG. 21a illustrates another embodiment of a loading system (410) of the present invention for loading a subject connector (collar and fitting) onto a graft vessel. Loading system (410) includes a base (412), an outer frame (collar) cartridge (414), an inner frame (fitting) cartridge (416) and a mating cartridge (418) (see FIG. 21b) combined into a single assembled tool. Base (412) has a mounting means, here a recessed slot (420), within its bottom surface to allow it to be mounted to a corresponding mounting means, such as a rail, of a retractor (not shown) or other stabilized instrument. The close proximity between a retractor or stabilizer-mounted loading system allows the system to be used with pedicled arteries (e.g., the LIMA and RIMA) or proximally connected vein grafts which reside within the chest. Outer frame cartridge (414) may be provided with a preloaded collar. Similarly, inner frame cartridge (416) may be provided with a preloaded fitting. Additionally, outer frame cartridge (414) may be provided preloaded or integral with base (412) prior to packaging of the system thereby eliminating at least one additional step in the above-described procedure. Mating cartridge (418), which may also be provided preloaded with outer frame cartridge (414) and/or base (412), includes pins (422) for holding the ears of the outer frame while the graft and inner frame are being coupled to it. Mating cartridge (418) facilitates automatic collar-to-graft mating and fitting-to-collar mating thereby eliminating the need for the user or physician to directly manipulate (e.g., by means of graspers or the like) either the collar or the fitting (i.e., only the graft vessel is manually manipulated). A plurality of interchangeable mating cartridges having varying sizes to accommodate varying sizes of connectors may be provided, thereby allowing use of the same loading system with various sized connectors. Additionally, mating cartridge (418) may be equipped with keyway grooves (424) for touch-less transfer of a loaded connector-graft assembly from loading system (410) to operative engagement with a deployment tool 168 of the present invention (see FIG. 21c). As such, various steps of the above-described connector-graft loading procedure are eliminated, thereby hastening the anastomosis process. Moreover, the risk of physician or user error is substantially minimized.

It is preferred that connector (4) be set and prepared for deployment within a deployment device, as shown in FIGS. 14a and 14b, before taking invasive action at the target site for an angled anastomosis. Regardless, an angled anastomosis site is prepared by creating an initial puncture, for instance, with the tip of a number 11 blade scalpel. Next, this opening is preferably extended longitudinally with scissors to about 3 mm to 7 mm in length depending on the connector size and anastomosis angle. Most often, a longitudinal slit of about 5 mm is preferred for a 30 degree, 3 mm connector. Scissors are advantageously provided in connection with an instrument. Otherwise, standard Potts scissors may be used. In one arteriotomy (or venotomy) instrument embodiment, a marker pen is used to place biocompatible ink on a marking instrument with a specified length and the marking instrument is used to tattoo an identifier as to the desired incision length. This helps direct the operator to cut the incision to the appropriate length without requiring the use of a specific blade instrument designed to only create the desired incision with a single actuation.

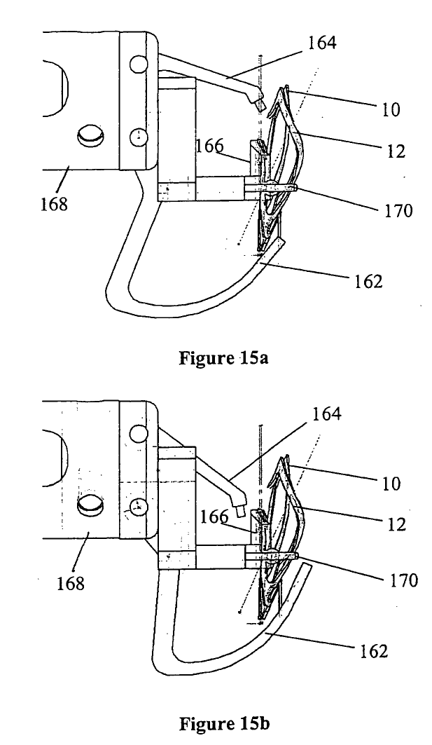

The deployment tool in FIGS. 14a to 14d, and 15a and 15b provides an end effector (168) which incorporate pins (170) that engage the ears (37) of the collar. This provides stabilization of the connector relative to the deployment tool and provides a reference from which to deflect the distal band (39) of the collar. It should be noted that the deployment tool may alternatively incorporate a clamping or other grasping mechanism to engage the base of the collar and/or fitting without having to penetrate components of either the collar or fitting. One such component is a stabilization platform (166) incorporated within the end effector (168) and configured to engage the front and/or lateral surfaces of the connector to maintain the position of the connector during deployment. A combination of stabilization platform (166) and pins (170) are used in the embodiments shown in FIGS. 14a to 14d, and 15a and 15b.

The deployment tool also incorporates a toe deflector (164) and a heel deflector (162) pivotally connected to end effector (168). During the deployment procedure, the toe deflector is used to engage the elliptical loop (31) of the collar to deflect and release the distal band (39) of the collar. The heel deflector is used to engage and deflect the trailing section (18) of the fitting during deployment. FIG. 15a shows the toe deflector (164) and the heel deflector (162) in the loading or release state. FIG. 15b shows the toe deflector (164) and the heel deflector (162) in the actuated state, ready for deployment of the connector. It should be noted that in FIG. 15b, the components of the connector are not shown deflected; in operation, movement of the toe deflector and heel deflector cause their counterparts on the connector to correspondingly deflect for deployment.

Once deployed, the heel deflector (162) and toe deflector (164) are released enabling the trailing section (18) of the fitting and the distal band (39) of the collar, respectively, to return towards their resting configuration causing the tissue (host vessel and graft) residing between the fitting and the collar to be compressed, like a gasket, and ensure hemostasis at the anastomosis. It should be noted that the toe deflector (164) and the heel deflector (162) may be actuated simultaneously; the toe deflector may be offset from heel deflection to enable full deployment of the trailing section of the fitting prior to full release of the distal band of the collar; or may be operated independently.

With the trailing segment and the distal band deflected into the deployment configuration, connector (4) is positioned into the host vessel. This is preferably performed by inserting the leading section (16) through the arteriotomy (or venotomy if the host vessel is a vein), and then advancing the lateral features (20) of fitting (10) as may be provided. Deflected trailing segment (18) is then advanced through the heel end of the arteriotomy and into host vessel (8); then the trailing segment (18) is released by actuating the deployment tool towards its resting configuration, as shown in FIG. 2, in order to secure the connector. Particularly in those variations of the invention as described above where movement of trailing segment articulates side portions (20), movement of trailing segment (18) to an host-vessel engaging position will also cause affected side portions (20) to engage the sides of host vessel (8) to maintain connector (4) in place.

In instances when a collar (12) is used in connector (4), it is also released to compress toe portion (48) of graft (6) against host vessel (8). Release of collar (12) may also result in compressing graft (6) against portions of host vessel (8) opposed by lateral fitting portions (20), especially when the lateral portions are integrated with the trailing segment.

The deployment tool embodiment shown in FIGS. 14a to 14d enables offsetting the movement of the toe deflector (164) relative to the heel deflector (162) with a single actuation mechanism. This offset facilitates full release of the trailing segment (18) prior to release of the distal band (39) of the collar with a single handle actuation to provide operator control of the connector release. As such the trailing segment (18) may be fully released so the operator can confirm its position within the host vessel, ensure the sides of the incision through the host vessel are appropriately positioned around the lateral portions (20) of the fitting, and/or de-air the graft prior to releasing the collar distal band (39).

The embodiment in FIGS. 14a to 14d includes two handle segments (146) rotatably connected to a handle block (142) at a proximal end directly with pins (156). The handle segment (146) is secured to linkages (148) that pass through slots in the handle block (142) at a mid-section and are secured to a rod (152) that contains a luer end (144) and a flush path (140). The flush path, as shown in FIGS. 14c and 14d provides a conduit for flushing cleaning solution, saline, or other fluid when cleaning the deployment tool, and/or injecting saline or CO₂ mist to clear the field of view from blood. The rod (152) moves within a shell (150) that is bonded to the handle block (142). The length and orientation of rod and shell are determined by the procedure specifics. For less invasive access, the rod and shell are relatively long (>15 cm) to ensure the connector may reach the host vessel without the handle segments (146) interfering with the access points into the patient. The rod and shell may be curved to enable changing the angular pathway for inserting the connector into the host vessel. Alternatively, the rod and/or shell may be made malleable to enable the operator to tailor the deployment tool to his/her access viewpoint.

A compression spring (154) provides resistance to advancing the rod (152) relative to shell (150) and handle block (142) and ensures the resting position of the deployment tool is in the deflected state. The compression spring (154) is stiff enough such that with the trailing segment (18) of the fitting and the distal band (39) of the collar deflected, the deployment tool may be handed to the operator without having to manually hold the handle apart or worrying that the handle may accidentally become actuated and release the connector before it is appropriately positioned. Alternatively, a locking mechanism may be incorporated in the deployment tool to ensure the handle does not accidentally actuate.

The stabilizer (166) is bonded to the shell (150) and provides a support for the connector and defines the pivots for the toe deflector (164) and the heel deflector (162). The stabilizer also determines the angle at which the connector sits relative to the rod and shell of the deployment tool. For reverse insertion the stabilizer (166) is configured to orient the toe of the connector at an acute angle (<90 degrees) to the shell of the deployment tool. For perpendicular insertion, the stabilizer is configured to orient the toe of the connector at approximately 90 degrees to the shell. For acute insertion, the stabilizer is configured to orient the heel of the connector at an acute angle (<90 degrees) to the shell.

The toe deflector (164) and the heel deflector (162) are rotatably attached to the stabilizer (166) with pins (156). Intermediate linkages (158 and 160) connect the proximal ends of the heel deflector (162) and the toe deflector (164) to the rod (152) with a second compression spring (154) to orient the deflectors in the appropriate resting, “deflected” orientation when released. The intermediate linkages (158 and 160) and the associated compression spring (154) enable the offset deflection of the toe deflector (164) from the heel deflector (162). As the heel deflector is actuated by squeezing the handles (146) the toe deflector (164) remains in the deflected, non-released position until the trailing segment (18) is fully released and the compression spring (154) is fully actuated such that movement of the rod engages the toe deflector linkage (160) which initiates the actuation of the toe deflector (164) and releases the distal band (39) of the collar. This two-staged release provides one additional benefit in that a tactile signal indicates the complete release of the trailing segment (18) and initiation of the release of the distal collar band (39). The toe deflector (164) provides another benefit in that it separates the ears (37) of the connector from engagement with the pins (170) once fully actuated to fully release the connector from the deployment tool and indicating completion of the angled anastomosis.

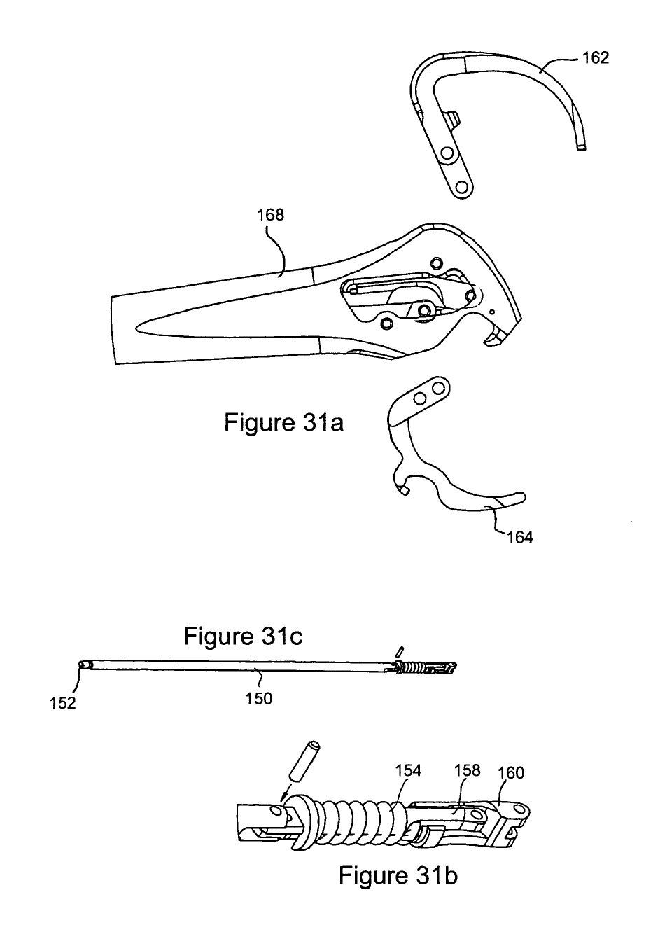

FIG. 31a illustrates another embodiment of an end effector (168) of a deployment tool having a toe deflector (164) and a heel deflector (162). Intermediate linkages (158 and 160) connect the proximal ends of the heel deflector (162) and the toe deflector (164) to the rod (152) with a compression spring (154) to orient the deflectors in the appropriate resting, “deflected” orientation when released. The intermediate linkages (158 and 160) and the associated compression spring (154) enable the offset deflection of the toe deflector (164) from the heel deflector (162). These linkages enable a two-stage deflection process where the heel deflector is released first and the toe deflector is offset from the heel deflector by the linkages to release after the heel deflector has completely released the trailing segment of the coupled graft.

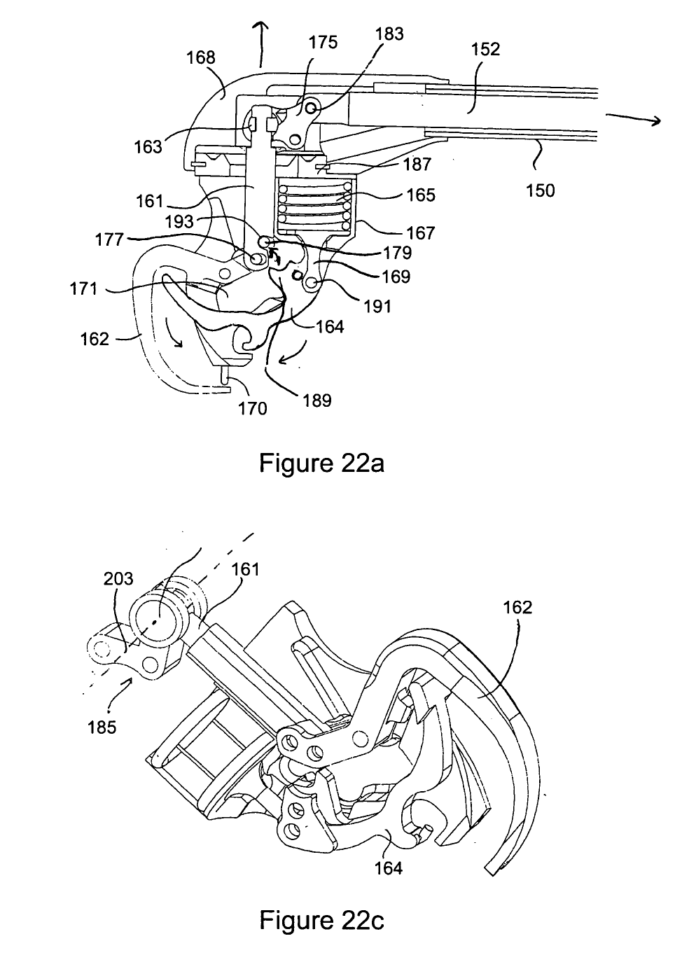

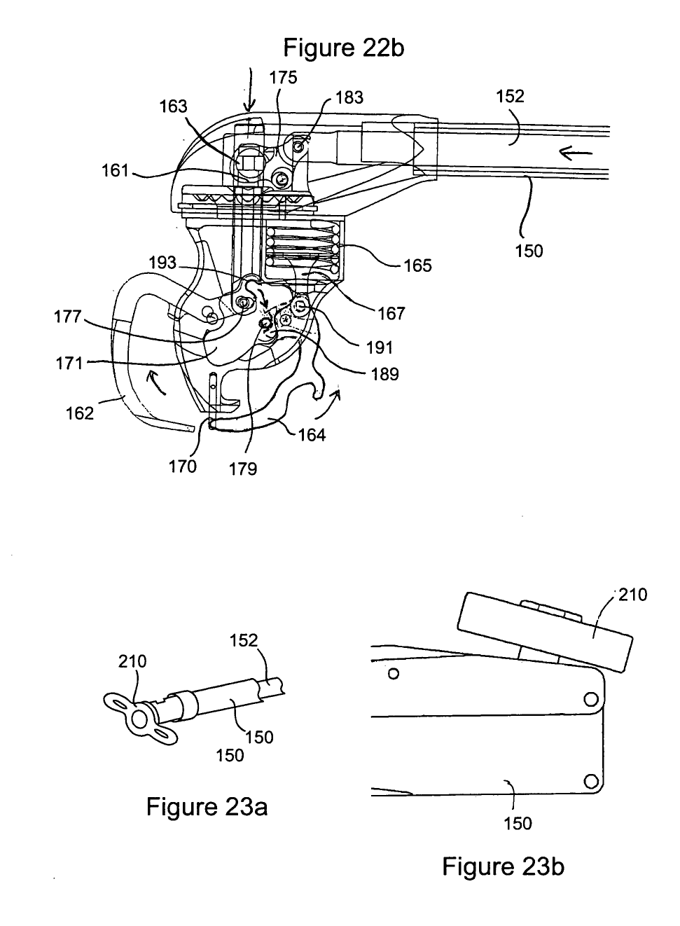

FIGS. 22a, 22b and 22c illustrate exposed views of the distal end of another embodiment of a deployment tool having an end effector (168) which is positioned at a right angle to the axis of shell (150) and rod (152). A combination of gears, rods and pins within the end effector allow the heel deflector (162) and the toe deflector (164) to be rotatably attached to and movable relative to the end effector while being linearly actuated by rod (152). An axial rod (152) is attached to a gear mechanism (175) by way of pin (183). A transverse rod (161), which controls the movement of heel deflector (162), is attached at a proximal end to gear (175) by way of a yoke pin (163) and is attached at a distal end to heel deflector (162) and another gear mechanism (171) by means of a pin (177). A piston (187) is also pivotally attached to gear mechanism (175) on the side opposite that of transverse rod (161) and resides over and engages a popette (167) having a spring (165) which in turn engages. Popette (167) is pivotally attached at a distal end (169) to toe deflector (164) by means of a pin (191). A pin (179) extends from gear mechanism (171) and defines a travel path between a pin stop (193) within transverse rod 161 and pin stop (189) of toe deflector (162) during actuation of the deployment tool.

FIG. 22a illustrates the end effector in a closed or advanced state wherein deflectors (162, 164) are drawn inward towards each other. In such a closed state, axial rod (152) is in a proximally retracted position causing transverse rod (161) to be in an upwardly biased position thereby maintaining heel deflector (162) in an inward or closed position. Additionally, gear (171) is caused to be biased in a counterclockwise rotation such that pin (179) resides within pin stop (193). Further, spring (165) is in its uncompressed or outwardly biased state, thereby pushing popette (167) downward thereby causing toe deflector (164) to be in an inward or closed position.

Actuation of the deployment tool to open or egress deflectors (162, 164) so that a subject connector may be grasped for deployment into a vessel involves the following actions with reference to FIG. 22b. As axial rod (152) is translated distally within shaft 150, gear mechanism (175) is caused to rotate counterclockwise, and by way of its configuration and attachment to yoke pin (163), pushes downward on transverse rod (161) thereby causing heel deflector (162) to rotate away from toe deflector (164). Additionally, gear mechanism (171) is caused to rotated clockwise wherein pin (179) travels from within pin stop (193) of transverse rod (161) to engage with pin stop (189) thereby forcing toe deflector (164) in a direction away from heel deflector (162) (commonly referred to as a “follower” action). The counter-clockwise force placed on toe deflector (164) is resisted (and the egress motion of the toe deflector is initially prevented) by an opposite or clockwise force applied by popette (165). The magnitude of the clockwise force is such that toe deflector (164) does not begin to open or egress until heal deflector (162) has rotated sufficiently. The amount of travel or rotation by which the heel deflector prior to travel or rotation by the toe deflector is selected according to the particular application and depends on the ratio between the height of the coupled graft and the length of the coupled graft but is typically in the range from about 30° to about 90° and in the embodiments shown above is in the range from about 60° to about 65°. Moreover, once toe deflector (164) begins its egress away from heel deflector (162), the rate at which the toe deflector rotates is slower than that of the heel deflector. Typically, heel deflector (162) travels at a greater rate than toe deflector (164). In this way, the deflectors may be controlled in a staged or phased manner. The ability to control the extent of egress by the deflectors minimizes the risk of stretching or over expanding the arteriotomy upon deployment of a connector.

An additional mechanism, such as adjustment knob (210) as illustrated in FIGS. 23a and 23b, may be employed with the subject deployment tools to provide additional control and additional fine-tune adjustment of the egress (opening) and advancement (closing) of the deflectors. Adjustment knob (210) is mated within the proximal end of shaft (150) of the deployment tool, for example, by means of a threading arrangement. Knob (210) may be provided in an end arrangement, such as illustrated in FIG. 23a or a side arrangement such as illustrated in FIG. 23b. The threading arrangement has a relatively high thread pitch, e.g., 40-50 threads per inch, wherein only minor torques of the knob (210) produce correspondingly slight axial advancement of the rod (150). In this way, the maximum advancement and/or egress positions of the heel deflector may be set. Because the torque-advancement resolution is so high, even variations in the graft wall thickness can be accommodated.

The deployment end effector of FIGS. 22a-22c have the further ability, by means of the engaging relationship between transverse rod (161) and yoke pin (163), to be manually rotated up to 360° about the axis defined by transverse rod (161). Additionally, as illustrated in FIG. 22c, the end effector may be configured to have the ability to be articulated about an axis (203) perpendicular to both axial rod (152) and transverse rod (161). Still yet, shaft (150) and rod (152) may be made of malleable or flexible materials which would provide maximum versatility in accessing a variety of anastomosis sites.

The loading and deployment tools described above may be provided as a cooperative system. For example, the loading tool of FIG. 21a may be used with the deployment tool of FIGS. 22a-22c whereby a mating cartridge (418) (see FIG. 21b) allows a hands-free approach to loading a connector onto a graft and then transferring the connector-graft assembly to the deployment tool. The latter step is accomplished by opening or egressing the deflectors or jaws of the end effector of the deployment tool, positioning the deployment tool relative to the mating cartridge (engaged on the loading tool) and operatively engaging the heel deflector 162 to the trailing segment (18) of the inner frame (10) of the connector and operatively engaging the toe deflector (164) to the grasping loop (31) of the outer frame (12) of the connector. Once properly engaged with the connector, the mating cartridge is released from the loading tool and is operatively attached to the deployment tool with the connector in position for delivery to and deployment at an anastomosis site as described previously.

Once in place, the completed anastomosis is inspected for leakage. This may be done before and/or after an anastomosis at the other end of the graft (if required) is complete. At a minimum, an inspection of the angled anastomosis should be made when blood is flowing through graft (6). If leakage is detected, and it cannot be remedied by adjustment of the graft or collar, the anastomosis site may be packed until bleeding terminates, bioglue (e.g., as available through Cryolife in Kennesaw, Ga.) may be applied to the anastomosis, and/or a stitch of suture material may be applied. In extremely rare instances where these steps do not prove adequate, it may be necessary to reposition or remove the connector (4).

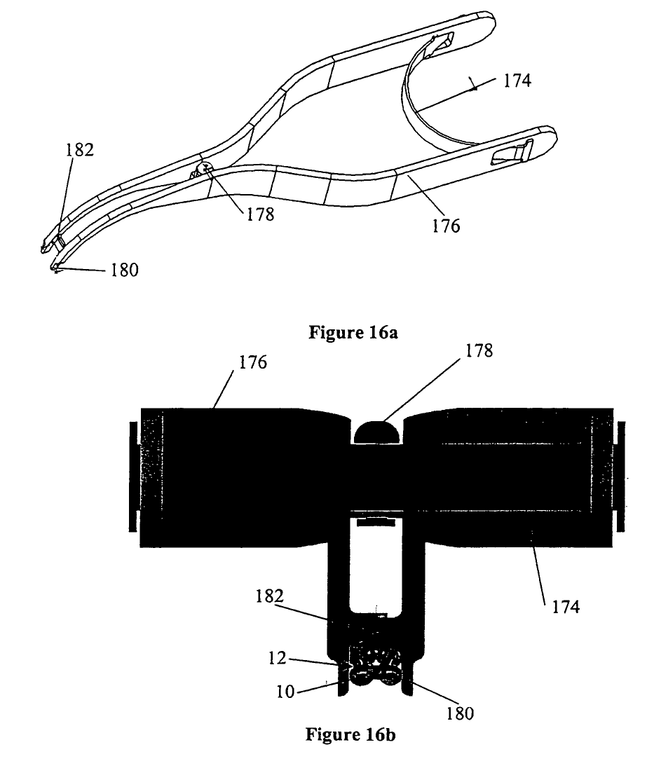

FIGS. 16a and 16b show a repositioning tool designed to spread the sides of the collar distal band (39) and manipulate the connector such that tissue enters the gap between the lateral portions (20) of the fitting and the distal band (39) of the collar. Once repositioned, the repositioning tool releases the collar. The repositioning tool has two handles (176) rotatably joined at a pivot pin (178) and with a spring (174). The functional end of the repositioning tool contains extensions (180) designed to fit within the edges of the distal band (39) and spread the distal band once actuated. A stabilization bar (182) is integrated with the extensions (180) and provides a surface to advance the connector once the distal band is spread open.

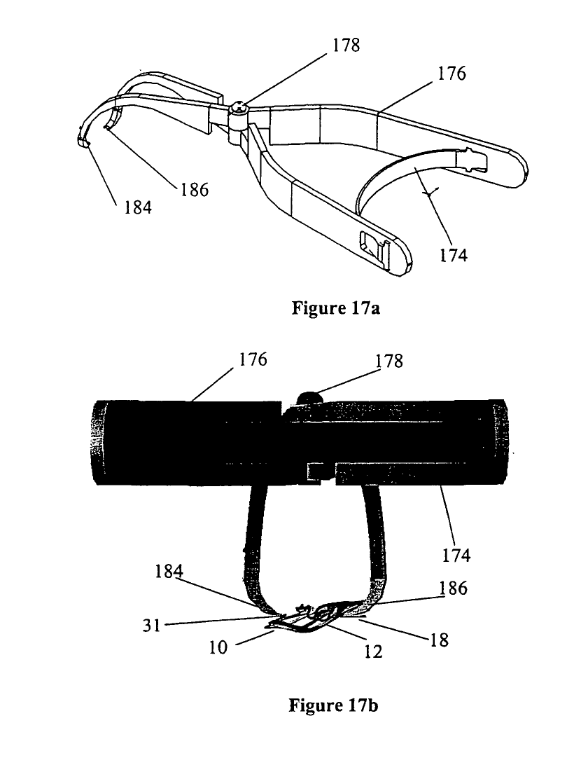

FIGS. 17a and 17b show an extraction/repositioning tool whose active end contains a toe-grasping rod (184) and a heel pusher (186) having similar engagement features as the toe deflector and heel deflector discussed above. The toe-grasping rod deflects the distal band (39) of the collar while the heel pusher deflects the trailing segment of the fitting. This tool may be used to partially deflect the distal band and trailing segment to reposition the connector or fully deflect those components to remove the connector from the host vessel.

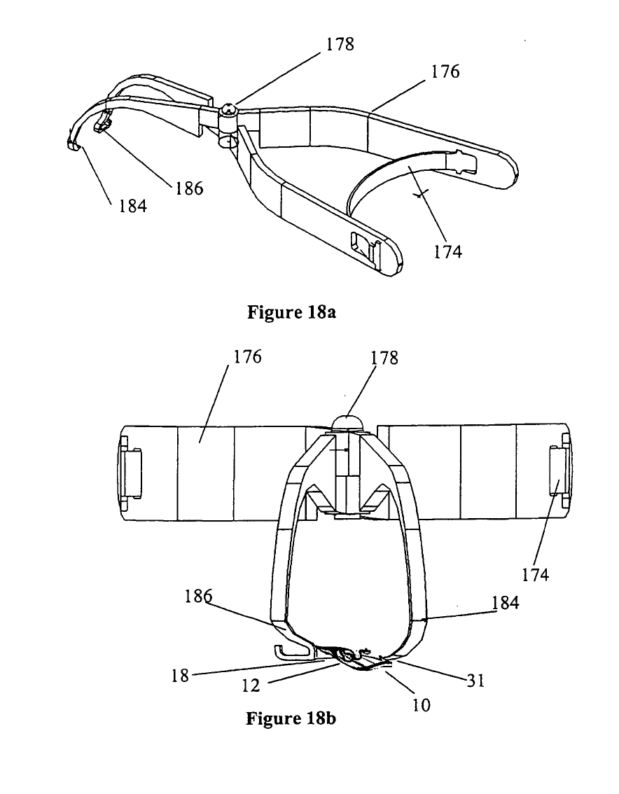

FIGS. 18a and 18b show a removal tool that differs from the embodiment in FIGS. 17a and 17b in that the heel pusher (186) is curved to fully advance the trailing segment (18) of the fitting as the curved end is advanced into the wedge between the base (14) of the fitting and the trailing segment (18).

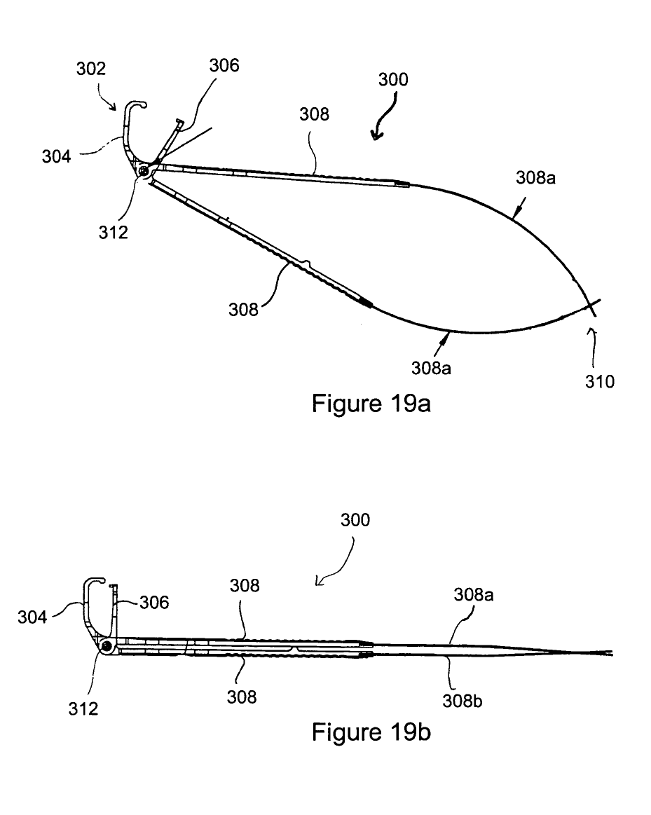

FIGS. 19a and 19b show another removal tool (300) having an end effector (302) comprised of jaw arms (304, 306) pivotably attached to handles (308) at pivot pin (312). Jaw arm (304) functions to deflect the heel of a connector of the present invention and jaw arm (306) functions to deflect the toe of the connector. Handles (308) have flexible proximal portions (308a and 308b) which are joined at their proximal end (310) to form a leaf spring mechanism. When handles (308) are in an outwardly biased position, the jaw arms are in an open position (as shown in FIG. 19a). When squeezed or compressed together jaw arms (304 and 306) are caused to close (as shown in FIG. 19b), and thereby deflect the leading segment of the outer frame, and the trailing segment of the inner frame, respectively, of the. End effector (302) while shown in a right angle configuration with respect to handles (308) may also be configured to be coaxial with the handles. Removal tool (300) may also be provided with an end effector which is rotatable about pivot pin (312) and adjustable as needed for accessing an implanted connector.

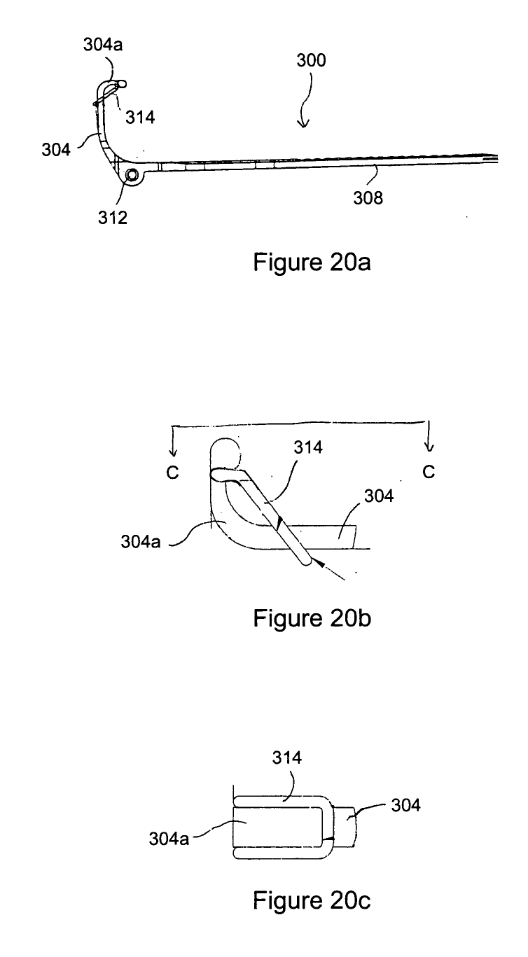

Jaw arm (302) of tool (300) may also be provided with a guide (314) as illustrated in FIGS. 20a, 20b and 20c to prevent slippage of a connector's trailing segment upon deflection during removal. Guide (314) may have any suitable configuration and is illustrated here as a rectangular crossbar extending across the distal bend or elbow (304a) of jaw arm (304). As the toe jaw arm 306 is engaged into the grasping loop of the outer frame, the heel jaw arm 304 of the removal tool is placed in the wedge between the trailing segment of the inner frame and the base of the coupler at the heel. The guide 314 prevents lateral slipping of the base of the coupled graft at the heel relative to the heel jaw arm 304 of the removal tool as the trailing segment is deflected. Once the trailing segment is fully deflected by the heel jaw arm 304, the coupled graft is released from the host vessel without causing abrasion or tearing of the host vessel.

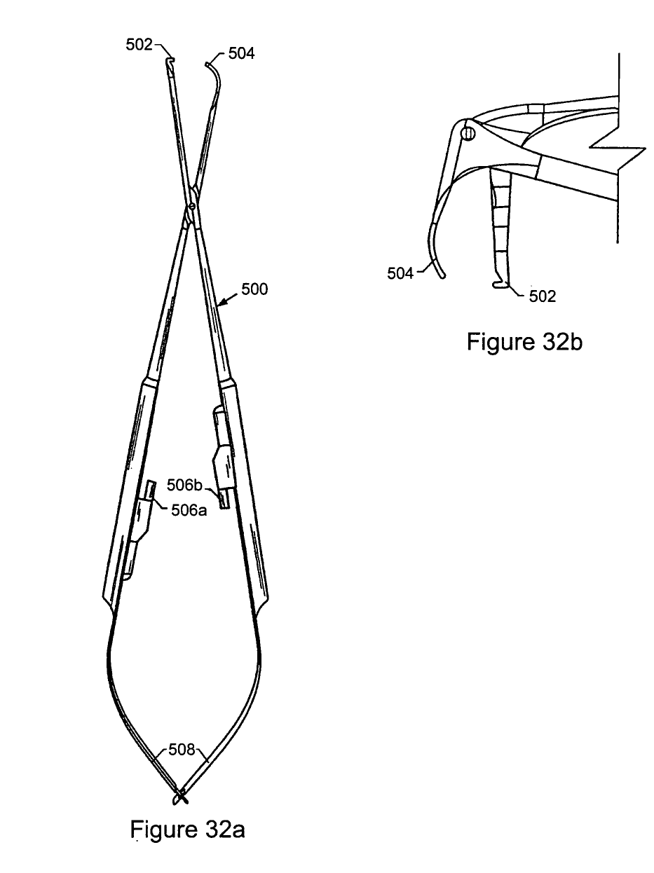

FIGS. 32a and 32b show another device which may be used to deploy and remove a connector of the present invention. Device (500) similarly adapted to draw back band of an outer frame while advancing rear segment of the inner frame. An interface section (502) captures the distal band while hook (504) advances the rear segment. To accommodate differences in anatomical access locations or paths, it is also possible to orient the end of the deployment device shown in FIG. 32a at another angular orientation as shown in FIG. 32b. In this case, the instrument head is shown rotated approximately 90°. It is also noted that device (500) optionally includes interlocking members (506a, 506b) and sprung arms (508), that work in conjunction with each other to provide a more stable, user-friendly device to maintain a connector in a state ready for deployment.

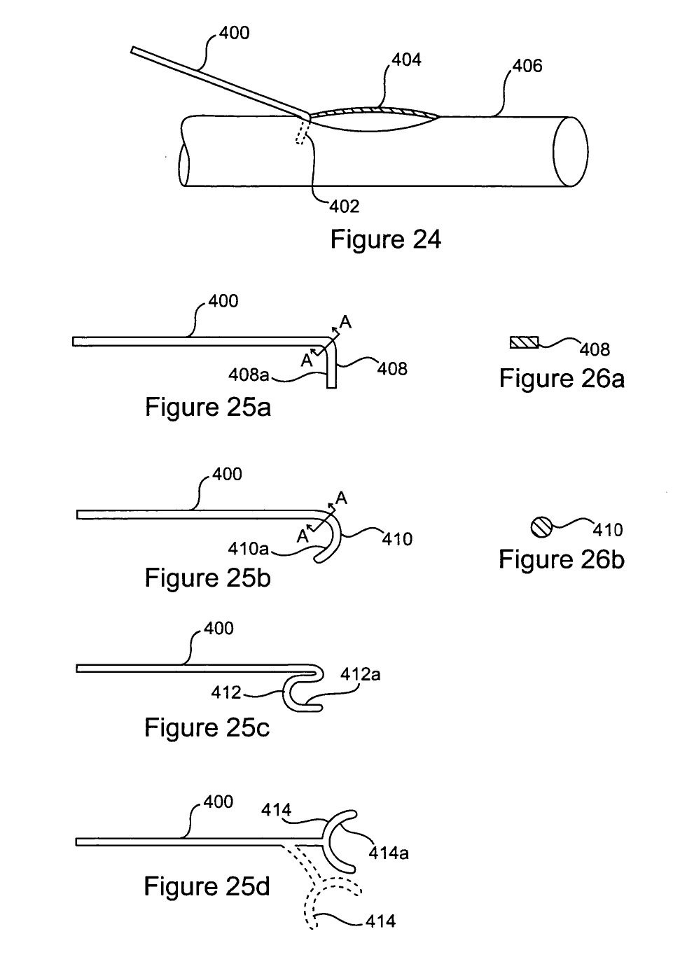

The above-described tools greatly facilitate forming an anastomotic connection between vessels, particularly in less invasive approaches where the surgical spaces are relatively small. Consequently, the surgeon is often working from a position or angle in which visualization of the anastomotic site is less than optimal. This can be particularly problematic when the coronary vessel being bypassed is one which is located more laterally and/or posteriorly on the heart, e.g., circumflex, obtuse marginals, posterior descending artery, and/or which is relatively deep within the myocardium. In particular, accurate placement of the heel or trailing segment of the inner frame of the connector within the arteriotomy may be extremely difficult. The visualization tools illustrated in FIGS. 24-28 address these issues whereby they are useful in enhancing visualization as well as access and delivery of the subject connectors, as well as removal of the connectors, if necessary.

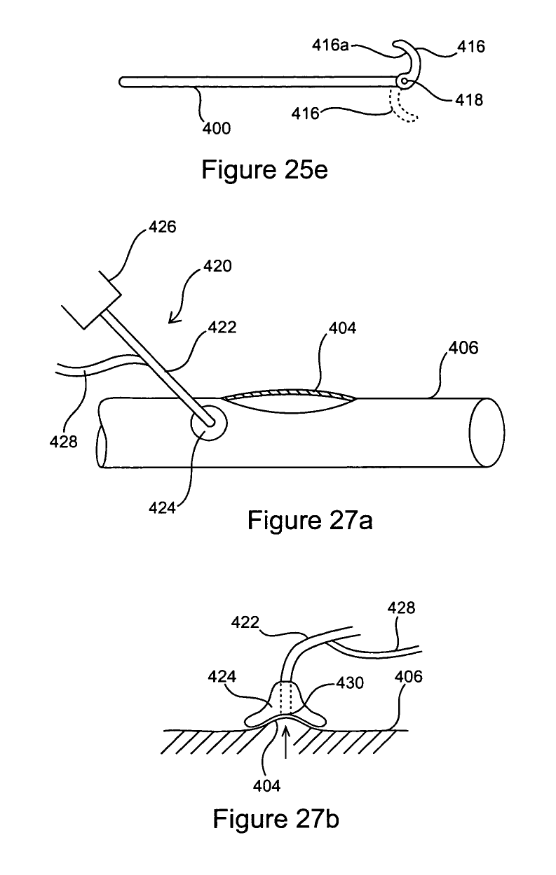

FIGS. 24 and 25 illustrate variations of “pushers” and “pullers” designed to capture an edge, particularly at the apex, of an arteriotomy (404) within the host vessel (406) during the deployment of the connector, as illustrated in FIG. 24. The pusher and pullers include a shaft (400) for proximal handling and manipulation by the physician and a distal working end. The embodiments of FIGS. 24, 25a, and 25b are “puller type” visualization tools having a working end where the “open” end or tissue-contacting surface is facing proximally. The variation shown in FIGS. 24 and 25a provides a straight segment (402) extending laterally at substantially a right angle to shaft 400. The tissue-contacting surface (408a) faces proximally toward shaft (400). FIG. 25b illustrates a variation of a puller having a curved or hooked distal working end (410) where the tissue-contacting surface (410a) is also facing the shaft. FIGS. 25c and 25d illustrate “pusher type” visualization tools where the “open” end or tissue-contacting surface of the end effector is facing distally or away from shaft (400). The end effector (412) of FIG. 25c has a C or U shaped structure displaced laterally from shaft 400 and having a tissue-contacting surface (412a) facing distally. The end effector (414) of FIG. 15d also has a C or U shaped configuration having a tissue-contacting surface (414a) facing distally, however, end effector (414) is positioned centrally wherein the legs of the structure define a symmetrical configuration about shaft (400). The end effector (416) of FIG. 25e has a curved configuration similar to that of end effector (410) in FIG. 25b, however, it has the additional feature and ability to rotate about an axis perpendicular to shaft (400), and thus, is adjustable between a puller type configuration and a pusher type configuration (shown in phantom). The range of motion of the end effector may be as much as 360° to provide the most versatility. The visualization tools described above may be made of a malleable material, as illustrated in FIG. 25d, wherein the shaft and/or the end effector can be bent or formed as necessary to accommodate a particular application. Further, the cross-sectional shapes of the end effectors may have any suitable shape and configuration. For example, as illustrated in FIGS. 26a and 26b, respectively, the cross-section may have a rectangular or circular shape. Additionally, the tip of the end effectors may have varying diameters or widths along their lengths. For example, the tip may taper distally or may increase in size distally. While only several shapes and configurations of end effectors have been illustrated and described, those skilled in the art will appreciate that there exist many other variations that are useful for facilitating visualization.

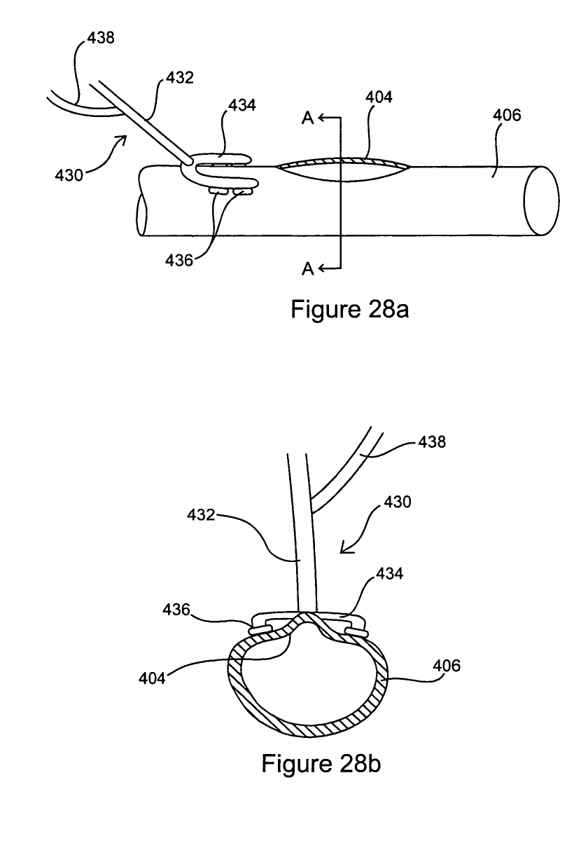

FIGS. 27 and 28 illustrate suction-based visualization tools. For example, the tool (420) of FIGS. 27a and 27b has a shaft (422) having an end effector (424) having at least one suction port (430) in its bottom surface. Port (430) is in fluid communication with a negative pressure source (not shown) by way of an airway passage which extends through end effector (424) and shaft (422) and through tubing (428). When negative pressure is applied, tissue present at port opening (430) is pulled upward, as illustrated in the cross-sectional view of FIG. 27b taken along line A-A. As such, the tool may be used to manipulate or tension the vessel wall (406) adjacent an arteriotomy site (404) so that it may be lifted, stretched or moved to create sufficient space for insertion of a segment of a connector without damage to the tissue. The suction-based end effectors may have any suitable configuration with any number of suction ports. FIGS. 28a and 29b illustrate a visualization tool (430) having a U-shaped end effector (434) having a plurality of suction ports (436) on the bottom surface of each foot of the end effector (434). The suction ports are in communication with a negative pressure source via tubing (438) and an internal channel within shaft (432) and through the feet of the end effector. Such a configuration allows a larger area or portion of the perimeter of arteriotomy (404) to be elevated, tensioned or stretched to accommodate passage of a connector.

For less invasive approaches, bridging or endoscopic vein harvesting tools may be utilized to access the host vessel, expose the host vessel and stabilize the host vessel as the arteriotomy is created and the connector is deployed into the host vessel. Such devices include the SaphLITE® manufactured by Genzyme Surgical, Inc. for saphenous vein harvesting. This, and other such bridging devices, may be used to access peripheral host vessels through a small incision, and enable a less invasive approach to inserting angled connectors into the popliteal artery, femoral artery, iliac artery, etc. due to the features of the connector and accessory devices. The connector may also be used in conjunction with anastomosis isolation devices such as the enclose® Anastomosis Assist Device manufactured by Novare Surgical, Inc. Such isolation devices clamp a region of the aorta and provide a membrane to prevent bleeding while the anastomosis is created. As such, the angled connector embodiments in this invention may readily be inserted through an incision created prior to or after deploying such isolation device and used to create the anastomosis.

Fabricating Connector Components

Now, returning to the elements of connector (4), optional inventive features and a manner of manufacture is described. A preferred manner of producing connector components according to the present invention is by machining tubing to include features that may be stressed and set into shape to produce connector elements like those depicted in FIGS. 1, 2, 3a, 3b, 4a, 4b, 6a, 6b, 6c, 6d, 7a, 7b, 8a, 8b, 9a, 9b 29a-29d and 30a-30d. Shapes so produced may be referred to as wireforms.

The machining may be accomplished by electron discharge machining (EDM), mechanically cutting, laser cutting or drilling, water-jet cutting or chemically etching. It is to be noted that portions of the connectors may be fabricated as a separate components and bonded by spot welding, laser welding or other suitable manufacturing process to form complete structures. Typically, after whatever cutting or forming procedure is employed, the material is set in a desired final shape. Where a metal is used, one or more flexure steps followed by heating will accomplish this. If the connector elements are made of alternate material such as a plastic or a composite, other forming procedures as would be apparent to one with skill in the art may be used.

Preferably, connector elements are made from a metal (e.g., titanium) or metal alloy (e.g., stainless steel or nickel titanium). Other materials such as thermoplastic (e.g., PTFE), thermoset plastic (e.g., polyethylene terephthalate, or polyester), silicone or combination of the aforementioned materials into a composite structure may alternatively be used. Also, connectors fabricated from nickel titanium may be clad with expanded PTFE, polyester, PET, or other material that may have a woven or porous surface. The fittings may be coated with materials such as paralyne or other hydrophilic substrates that are biologically inert and reduce the surface friction. To further reduce the surface tension, metallic or metallic alloy fittings may be bead blasted, chemically etched, and/or electropolished. Evidence suggests that electropolishing reduces platelet adhesion because of the smooth surface. Alternatively, the fittings may be coated with heparin, thromboresistance substances (e.g., glycoprotein IIb/IIIa inhibitors), antiproliferative substances (e.g., rapamycin), or other coatings designed to prevent thrombosis, hyperplasia, or platelet congregation around the attachment point between the bypass graft and the host vessel. Alternatively, a material such as platinum, gold, tantalum, tin, tin-indium, zirconium, zirconium alloy, zirconium oxide, zirconium nitrate, phosphatidyl-choline, or other material, may be deposited onto the fitting surface using electroplating, sputtering vacuum evaporation, ion assisted beam deposition, vapor deposition, silver doping, boronation techniques, a salt bath, or other coating process.

A still further improvement of the fittings is to include beta or gamma radiation sources on the end-side fittings. A beta or gamma source isotope having an average half-life of approximately 15 days such as Phosphorous 32 or Palladium 103 may be placed on the base and/or petals of the end-side fitting using an ion-implantation process, chemical adhesion process, or other suitable method. Further details as to optional treatments of connectors according to the present invention are described in 10.00. Of course, connector fitting (10) and any associated collar (12) may be made differently. To avoid electrolytic corrosion, however, dissimilar metals should not be used.

Preferably, NiTi (Nitinol) tubing or flat stock is used to produce connector components. Irrespective of material format, a preferred alloy includes a 54.5-57% Ni content, and a remainder Ti by weight (less minor amounts of C, O, Al, Co, Cu, Fe, Mn, No, Nb, Si and W) is used. Such alloy has an A_{f} for at about −10 to −15° C. Consequently, for typical handling and in use, the material will exhibit superelastic properties as is most desired.

Still, it is contemplated that connectors according to the present invention may utilize thermoelastic or shape memory characteristics instead, wherein the material of either or both fitting (10) and connector (12) change from a martensitic state to an austenitic state upon introduction to an anastomosis site and exposure to a sufficiently warm environment. Taking advantage of the martensitic state of such an alloy will ease deflecting rear segment (18) and distal band (39) and maintaining their positions until placement.

Utilizing either thermoelastic or superelastic properties makes for a connector that can have certain members stressed to a high degree and return without permanent deformation from a desired position. However, it is contemplated that either or both fitting (10) and collar (12) may be made of more typical materials such as stainless steel or plastic. For fitting (10), this is feasible in view of the manner in which rear segment (18) is displaced for insertion into a host vessel. Hinge section (28) permits designs in which the stress applied by torsion is lower that applied in simply deflecting a rear petal or segment as shown and described in U.S. and foreign patents and applications entitled, “Improved Anastomosis Systems”, U.S. patent application Ser. No. 09/730,366; “End-Side Anastomosis Systems”, PCT Publication No. WO 01/41653; “Advanced Anastomosis Systems (II)” U.S. patent application Ser. No. 09/770,560.

This being said, the tube stock used to prepare distal connector fitting preferably has an outer diameter between 0.080 and 0.240 in (2 to 6 mm) and a wall thickness between 0.004 and 0.010 in (0.1 to 0.25 mm). Slightly larger diameter stock (or end product) will be used for each matching collar. The stock thickness for NiTi material used to form collars will typically have a wall thickness between about 0.004 in and about 0.010 in. Especially, for fitting (10) where it is possible to use thin stock in view of strength requirements, this will be preferred in order to minimally obstruct blood flow past the fitting. Larger connector components will typically be made of thick stock to account for increased stiffness required of such configurations relative to smaller ones.

The invention has been described and specific examples or variations of the invention have been portrayed. The use of those specific examples is not intended to limit the invention in any way. In all, it is to be understood that each of the features described in connection with the various connector components and projections for forming the same may be mixed and matched to form any number of desirable combinations. Further, it is contemplated that additional details as to the use or other aspects of the system described herein may be drawn from Abstract, Field of the Invention, Background of the Invention, Summary of the Invention, Brief Description of the Drawings, the Drawings themselves and Detailed Description and other background that is intended to form part of the present invention, including any of the patent applications cited above, each of which being incorporated by reference herein in its entirety for any purpose. Also, to the extent that there are variations of the invention which are within the spirit of the disclosure and are equivalent to features found in the claims, it is the intent that the claims cover those variations as well. All equivalents are considered to be within the scope of the claimed invention, even those which may not have been set forth herein merely for the sake of relative brevity. Finally, it is contemplated that any single feature or any combination of optional features of the inventive variations described herein may be specifically excluded from the invention claimed and be so-described as a negative limitation.

What is claimed is:

1. A system for loading an anastomosis connector assembly onto a graft vessel, the connector assembly comprising an inner frame and an outer frame, the outer frame having an original state and an expanded state, the system comprising:
an outer frame cartridge configured for releasably holding the outer frame in the expanded state for placement over the graft vessel; and
an inner frame cartridge configured for releasably holding the inner frame and for operatively engaging the inner frame within the graft vessel when the outer frame is in the expanded state.

2. The system of claim 1, further comprising a platform, wherein the outer frame cartridge comprises a mechanism for interlocking the outer frame cartridge to the platform.

3. The system of claim 2, wherein the platform further is configured to receive the inner frame cartridge in apposition to the outer frame cartridge and to translate the inner frame cartridge relative to the outer frame cartridge.

4. The system of claim 2, further comprising a gauge for indicating the distance translated by the inner frame cartridge.

5. The system of claim 1, wherein the inner frame cartridge comprises a handle and a shaft extending therefrom wherein the distal end of the shaft is configured for releasably holding the inner frame connector.

6. The system of claim 1, further comprising a mating cartridge configured for releasable engagement with the outer frame and the inner frame upon operative engagement between the outer and inner frames.

7. The system of claim 6, wherein the mating cartridge is configured for engagement with a tool for deploying the interconnected connector assembly and graft at an anastomosis site.

8. The system of claim 6, wherein the mating cartridge is configured to engage with outer and inner frames having any suitable size.

9. A method for loading an anastomosis connector assembly onto a graft vessel, the connector assembly comprising an inner frame and an outer frame, the outer frame having an original state and an expanded state, the method comprising:
providing the outer frame releasably engaged to an outer frame cartridge wherein the outer frame is provided in the expanded state;
positioning at least a portion of the outer frame about the outside of the graft vessel;
providing the inner frame releasably engaged to an inner frame cartridge;
appositioning the inner frame cartridge with the outer frame cartridge wherein at least a portion of the inner frame is positioned within the graft vessel; and
releasing the outer frame from the expanded state to the original state.

10. The method of claim 9, further comprising releasing the inner frame from the inner frame cartridge.

11. The method of claim 9 or 10, wherein the graft vessel is securely fixed between the outer frame and the inner frame.

12. The method of claim 9, wherein the releasing the outer frame comprises interconnecting the outer frame with the inner frame.

13. The method of claim 9, further comprising mounting the outer frame cartridge to a stable platform prior to appositioning the inner frame cartridge.

14. The method of claim 13, further comprising mounting the outer frame cartridge to the stable platform prior to appositioning the inner frame cartridge.

15. The method of claim 14, further comprising using a means on the stable platform to expand the outer frame member.

16. The method of claim 14, further comprising using a means on the stable platform to apposition the inner frame cartridge with the outer frame cartridge.

17. An instrument for deploying an anastomosis connector assembly at an anastomosis site, the connector assembly comprising interconnected deflectable inner and outer frames, the instrument comprising:
an outer frame deflector; and
an inner frame deflector;
wherein the deflectors are configured to hold the interconnected anastomotic connector assembly, the outer frame deflector is configured to engage and selectively deflect a portion of the outer frame, and the inner frame deflector is configured to engage and selectively deflect a portion of the inner frame; and
wherein actuation of the deflectors advances the deflectors towards each other to deflect the frames and subsequently release the frames wherein the interconnected connector is deployed as the anastomosis site.

18. The instrument of claim 17, wherein the outer frame deflector and the inner frame deflector are independently operable.

19. The instrument of claim 17, wherein the outer frame deflector and the inner frame deflector are simultaneously operable.

20. The instrument of claim 19, wherein the instrument is configured wherein movement of the outer frame deflector is offset from movement of the inner frame deflector.

21. The instrument of claim 20, wherein the inner frame deflector releases the inner frame prior to the outer frame deflector releasing the outer frame.

22. The instrument of claim 21, wherein the movement of the deflectors is a rotational movement and wherein the outer frame deflector commences rotating after the inner frame deflector has rotated an angle in the range from about 30° to about 90°.

23. The instrument of claim 21, wherein the instrument further comprises:
an elongated shaft having a longitudinal axis;
a handle mechanism at a proximal end of the elongated shaft; and
an end effector at a distal end of the elongated shaft and comprising the deflectors, wherein the deflectors are movable by the handle mechanism.

24. The instrument of claim 23, wherein the end effector is rotatable about the shaft longitudinal axis.

25. The instrument of claim 23, wherein the end effector is rotatable about an axis other than the shaft longitudinal axis.

26. The instrument of claim 24, wherein the end effector is rotatable about an axis transverse to the shaft longitudinal axis.

27. A system for implanting an anastomosis connector assembly at anastomotic site to interconnect a graft vessel with a host vessel, the connector assembly comprising interconnectable frames comprising an inner frame and an outer frame, the outer frame having an original state and an expanded state, the system comprising:
a loading apparatus comprising an outer frame cartridge configured for releasably holding the outer frame in the expanded state for placement over the graft vessel, and an inner frame cartridge configured for releasably holding the inner frame and for operatively engaging the inner frame within the graft vessel when the outer frame is in the expanded state; and
a deployment apparatus comprising an elongated shaft and an end effector at a distal end of the elongated shaft, the end effector comprising first and second rotatable deflectors to configured to engage with and remove the interconnected connector assembly from the loading apparatus.

28. The system of claim 27, further comprising a mating cartridge configured for engagement with the loading apparatus and the deployment tool wherein and for transferring the

29. The system of claim 28, wherein the mating cartridge is configured to engage with outer and inner frames having any suitable size.

30. The system of claim 27, further comprising a selection of outer and inner frames of varying sizes.

31. A method for implanting an anastomosis connector assembly at anastomotic site to interconnect a graft vessel with a host vessel, the connector assembly comprising interconnectable frames comprising a deflectable inner frame and a deflectable outer frame, the outer frame having an original state and an expanded state, the method comprising:
expanding the outer frame into the expanded state;
positioning at least a portion of the outer frame about the outside of the graft vessel;
appositioning the inner frame with the outer frame wherein at least a portion of the inner frame is positioned within the graft vessel;
releasing the outer frame from the expanded state to the original state wherein the outer frame is interconnected to the inner frame;
deflecting the inner and outer frames;
inserting at least a portion of the deflected inner and outer frames into the host vessel; and
releasing the deflected inner and outer frames wherein the graft vessel is in fluid communication with the host vessel.

32. The method of claim 31 wherein the expanding, positioning, appositioning and first releasing steps comprise using the loading apparatus of claim D1.

33. The method of claim 32 wherein the deflecting, inserting and second releasing steps comprise using the deployment tool of claim D1.
